(19) United States
(12) Patent Application Publication
(10) Pub. No.: US 2010/0130416 A1
(43) Pub. Date: May 27, 2010

(54) MODULATION OF ANGIOGENESIS BY A-BETA PEPTIDE FRAGMENTS

(75) Inventors: Daniel Paris, Sarasota, FL (US); Michael J. Mullan, Sarasota, FL (US)

Correspondence Address: SENNIGER POWERS LLP, 100 NORTH BROADWAY, 17TH FLOOR, ST LOUIS, MO, 63102, US

(73) Assignee: ARCHER PHARMACEUTICALS, INC., SARASOTA, FL (US)

(21) Appl. No.: 12/471,279

(22) Filed: May 22, 2009

Related U.S. Application Data
(63) Continuation-in-part of application No. 11/598,299, filed on Nov. 13, 2006.
(60) Provisional application No. 61/055,394, filed on May 22, 2008.
(60) Provisional application No. 60/735,472, filed on Nov. 10, 2005.

(51) Int. Cl. A61K 38/16; C07K 7/06; C07K 7/08; C07K 14/00; C07K 7/50; A61K 38/08; A61K 38/10; G01N 33/53

(52) U.S. Cl. 514/12; 530/328; 530/327; 530/326; 530/325; 530/324; 530/317; 514/13; 514/14; 514/15; 514/16; 435/7.1

(57) ABSTRACT

Provided are Aβ peptide fragments that are useful in inhibiting angiogenesis. Also provided are methods for the treatment of pathological or unwanted angiogenesis and conditions and diseases associated therewith by administering an effective amount of an Aβ fragment. In a particular embodiment, the peptide fragment includes the sequence HHQKLVFF.

### FIELD OF THE INVENTION

The present invention is related to compositions and methods for treating diseases and pathological conditions or processes mediated by pathological angiogenesis by administering biologically active fragments of full length Aβ peptides to a patient suffering from such diseases, conditions, or processes.

### DESCRIPTION OF RELATED ART

Alzheimer's disease (AD) is the major cause of dementia in the elderly in Western countries, and is characterized by the progressive accumulation of intracellular neurofibrillary tangles, extracellular parenchymal senile plaques, and cerebrovascular deposits (Sissodia, et al. F.A.S.E.B. J. 9:366-370 (1995)). The principal component of senile plaques and cerebrovascular deposits is the β-amyloid peptide, the aggregated form of which consists of the 39-43 amino acid residue Aβ peptides that are proteolytically derived from the amyloid precursor protein (APP) (Naidu, et al. 1995 J. Biol. Chem. 270:1369-1374; Gorevic, et al. 1986 J. Neuropathol. Exp. Neurol. 45, 647-64; Selkoe, et al. 1986 J. Neurochem. 46, 1820-34). The primary protein component of senile plaques is beta/A4 amyloid, a 42-43 amino acid peptide.

Vascular pathology is the norm in advanced cases of AD, with cerebral amyloid angiopathy (CAA) being one of the most common abnormalities detected at autopsy (Ellis, et al. Neurology 46:1592-1596 (1996)). Certain vascular lesions, such as microvascular degeneration affecting the cerebral endothelium and periventricular white matter lesions, are evident in most AD cases (Ellis, et al. Neurology 46:1592-1596 (1996); Kalaria, Ann. N.Y. Acad. Sci. 893:113-125 (1999)). Furthermore, morphological alterations have been observed in AD brain microvessels and capillaries; in particular, terminal arterioles frequently have focal constriction and smooth muscle cells with an irregular shape and arrangement (Hashimura et al. Jpn. J. Psychiatry Neurol. 45:661-665 (1991)). Capillaries in AD brain typically show an abnormal abluminal surface with irregular constriction and dilatation along their paths (Kimura et al. Jpn. J. Psychiatry Neurol. 45:671-676 (1991)). Functional imaging techniques including positron emission tomography (PET) and single photon emission computerized tomography (SPECT) have revealed the existence of hypoperfusion in individuals prior to the time that they meet clinical criteria for AD suggesting that vascular abnormalities occur early during the disease process (Nagata et al. Neurobiology of Aging 21:301-307 (2000); Johnson et al. Neurobiology of Aging 21:289-292 (2000)). In other disorders involving cerebrovascular damage (such as traumatic brain injury, stroke and brain arteriovenous malformation), angiogenesis is a prominent response (Mendis et al. Neurochem. Res. 23:1117-23 (1998); Slevin et al. Stroke 31:1863-70 (2000); Hashimoto et al. Circ. Res. 89:111-3 (2001)). Given the plethora of reports on cerebrovascular damage in AD brain, the induction of an angiogenic reparative response would be expected, although there has been very little work in this area.

Several assays have been developed to study the specific steps involved in the angiogenic process (adhesion, migration, growth, invasion and differentiation). Knowledge of the effects of Aβ on angiogenesis would be of value in understanding its role in the micro-cerebrovascular abnormalities observed in AD. In the AD brain, Aβ peptides are known to form fibrillar deposits around blood vessels, leading to cerebral amyloid angiopathy (CAA) (Pardridge, et al. 1987 J. Neurochem. 49, 1394-401; Jellinger K. A., Attems J. 2005 J. Neurol. Sci. 229-230, 37-41). The increased levels of soluble and deposited Aβ in the AD brain can induce vascular damage, inflammation/gliosis, and reduced cerebral blood flow (Paris, et al. 2000 Ann. N.Y. Acad. Sci. 903, 97-109; Johnson, et al. 2005 Radiology. 234, 851-9). Numerous studies have shown that vascular functional impairments and reduced blood flow are characteristic features of the AD brain (Nicoll, et al. 2004 Neurobiol. Aging. 25, 589-97 and 603-4; Paris, et al. 2004 Brain Res. 999, 53-61; Beckmann, et al. 2003 J. Neurosci. 23, 8453-9; Farkasm, et al. 2001 “Cerebral microvascular pathology in aging and Alzheimer's disease” Prog. Neurobiol. 64, 575-611). Recently, it has been shown that angiogenesis is impaired in AD, and that this is associated with alterations in genes involved in vascular differentiation (Wu, et al. 2005 Nat. Med. 11, 959-65). A reduced brain capillary density is known in transgenic mouse models of AD (Paris, et al. 2004 Neurosci. Lett. 360, 80-5; Lee, et al. 2005 Brain Res. Bull. 65, 317-22). An impaired formation of capillary like structures on reconstituted basement membrane by endothelial cells and arterial explants harvested from the brains of TgAPPsw mice, suggesting abnormal alterations in the angiogenic response in TgAPPsw mice was recently demonstrated. (Paris, et al. 2004 Neurosci. Lett. 360, 80-5).

U.S. Patent Publication No. 2003/0077261 to Paris et al. discloses that Aβ peptides can be used as anti-angiogenic agents, and discloses the sequences of A-Beta peptides and APP as well as the nucleic acids encoding them, which are shown in the attached Sequence Listing shown in FIG. 10.

Angiogenesis is inhibited by Aβ peptides in multiple different in-vitro and in-vivo assays (Paris, et al. 2004 Angiogenesis. 7, 75-85). In-vitro, Aβ₁₋₄₀ and Aβ₁₋₄₂ can dose dependently inhibit capillary tube formation by human brain microvascular endothelial cells when plated on Matrigel, and can promote capillary degeneration at high doses. Mutants of the full-length Aβ peptide, including 1 or 2 amino acid substitutions, were also found to be biologically active anti-angiogenics. However at low doses, Aβ appears to be pro-angiogenic (Paris, et al. 2004 Angiogenesis. 7, 75-85; Cantara, et al. 2004 F.A.S.E.B. J. 18, 1943-5).

### SUMMARY OF THE INVENTION

It has been surprisingly discovered that biologically active fragments of full length Aβ peptides that have enhanced stability are useful as anti-angiogenic agents. These anti-angiogenic Aβ peptide fragments may be used to treat pathological conditions mediated by undesired and/or uncontrolled angiogenesis (characterized as “angiogenic diseases”), as described further herein.

Thus, in a first aspect, the present invention provides a variety of anti-angiogenic Aβ peptide fragments as well as compositions which include one or more such fragments that have been modified to increase stability or bioavailability. In one embodiment, the biologically active Aβ peptide fragment may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids in length.

In a particular embodiment, the anti-angiogenic Aβ peptide fragment is the Aβ₁₋₂₈ peptide fragment, the Aβ₁₀₋₃₅ peptide fragment, the Aβ₁₂₋₂₈ peptide fragment, the Aβ₁₃₋₂₀ peptide fragment, or other biologically active fragments or variants or homologs thereof.

In a specific embodiment, the anti-angiogenic Aβ peptide fragment is Aβ₁₂₋₂₈ and contains the amino acid sequence HHQKLVFF, or biologically active fragments, variants or homologs thereof.

In another specific embodiment, the anti-angiogenic Aβ peptide fragment is Aβ₁₃₋₂₀ or the amino acid sequence HHQKLVFF, or biologically active variants or homologs thereof. The variants may include, for example, amino acid substitutions.

In another embodiment, the Aβ peptide fragment comprises the amino acid sequence EVHHQKLVFF, or a biologically active fragment or variant thereof.

In another embodiment, the present invention is a pharmaceutical composition comprising an anti-angiogenic Aβ peptide fragment and one or more pharmaceutically acceptable carriers, diluents, or excipients.

In certain embodiments, the peptide fragment includes at least one modified amino acid. The fragment can also contain 2, 3, 4, or more modified amino acids. In certain embodiments, at least one amino acid has been modified by acetylation. In certain other embodiments, the peptide fragment includes at least one non-natural amino acid. In specific embodiments, the fragment comprises at least one D-amino acid. In specific embodiments, the peptide includes 2, 3, 4 or more D-amino acids. In specific embodiments, the peptide includes only D-amino acids.

In certain embodiments, the peptide has been modified by addition of a linker or other stabilizing molecule. In certain instances, the additional molecule can be a polyethylene glycol. In other instances, the additional molecule can include a cholesterol or other soluble polymer.

In a second aspect, the present invention provides a method for treating a disease or disorder mediated by pathological angiogenesis by administering to a subject in need thereof an effective amount of a biologically active Aβ peptide fragment, wherein the fragment is between 8 and 39 amino acids in length. The anti-angiogenic Aβ peptide fragment is optionally administered in combination or alternation with one or more therapeutic agents. The subject may be, for example, a mammal such as a human.

In one embodiment, the present invention is a method for treating cancer by administering to a subject in need thereof an effective amount of a biologically active Aβ peptide fragment, optionally, in combination or alternation with one or more chemotherapeutic agents.

In a particular embodiment, the present invention is a method of treating cancer by administering to a subject in need thereof an effective amount of a Aβ₁₂₋₃₈ peptide fragment containing the amino acid sequence HHQKLVFF or biologically active fragments, variants or homologs thereof.

In another particular embodiment, the method of treating cancer involves administering to a subject in need thereof an effective amount of Aβ₁₃₋₂₀ peptide fragment or the amino acid sequence HHQKLVFF or biologically active variants or homologs thereof.

The biologically active Aβ peptide fragment can be administered by any suitable means including, but not limited, to oral, parenteral, intravenous, intraarterial, pulmonary, mucosal, topical, transdermal, subcuteaneous, intramuscular, intrathecal or intraperitoneal administration.

A third aspect of the present invention provides diagnostic methods and kits for detection and measurement of anti-angiogenic Aβ peptide fragment activity in biological fluids and tissues.

A fourth aspect of the present invention provides diagnostic methods and kits to screen for compounds that are potentially therapeutic in treatment of Alzheimer's disease by interfering with the anti-angiogenic effect of the Aβ peptide fragment.

### DESCRIPTION OF DRAWINGS

FIG. 1 is a graph of the total length of capillary tubes expressed as a percentage of control treatment for 0, 1, 5 and 10 μM doses of various Aβ peptide fragments as described in Example 8.

FIGS. 2A and 2B are charts of the cellular proliferation and cellular adhesion of HUVEC samples, expressed as a percentage of the control, after incubation with various Aβ peptide fragments as described in Example 9.

FIG. 3 is a chart of the total length of capillary tubes expressed as a percentage of control treatment versus treatment with heparin (0.5 or 1 mg/ml), Aβ1-42 peptide, Aβ+heparin (500 μg/ml) and Aβ+heparin (1 mg/ml) as described in Example 10.

FIG. 4 is a graph of the total length of capillary tubes expressed as a percentage of control treatment for 0, 1, 5 and 10 μM doses of Aβ₁₋₂₈, Aβ₁₋₂₈ GGQGL and Aβ₁₋₂₈ AAQAL as described in Example 11.

FIG. 5 provides photographs (at 4× magnification) of capillaries tubes formed following incubation with Aβ peptide fragments as described in Example 11.

FIG. 6 is a graph of the total length of capillary tubes expressed as a percentage of control treatment for 0, 1, 5 and 10 μM doses of the peptides HHHQKLVFF, VHHQKLVII, and VHHQKLVKK as described in Example 12.

FIG. 7 is a chart of the Angiogenic Index (AI) for the rat corneal micropocket assay in response to 200 ng VEGF, VEGF+0.5 μg Aβ₁₂₋₂₈, VEGF+2.5 μg Aβ₁₂₋₂₈ and VEGF+5.0 μg Aβ₁₂₋₂₈ as described in Example 13.

FIG. 8 is a chart of the Angiogenic Index (AI) for the rat corneal micropocket assay in response to VEGF, 5 ug Aβ₁₋₂₈ GGQGL, and 0.5 ug, 2.5 ug and 5 ug of Aβ₁₂₋₂₈ and HHH-peptide (HHHQKLVFF), as described in Example 14.

FIG. 9 provides representative photographs of rat corneal micropockets following a seven day incubation as described in Example 14, including a VEGF control and 0.5 μg, 2.5 μg and 5.0 μg of Aβ₁₂₋₂₈.

FIG. 10 is a graph of the effect of the peptide EVHHQKLVFF on the growth of MCF-7 human breast tumor xenografts in nude mice over time after IP injection of either vehicle or 50 mg/Kg peptide fragment. The tumor sections were immunostained with a PECAM-1 antibody 42 days after the implantation of MCF-7 tumor cells in nude mice.

FIG. 11 shows pictures of PECAM-1 immunostaining (brown staining) of breast tumor sections after injection of vehicle (top row) or peptide EVHHQKLVFF 30 days post-tumor implantation. The tumor sections were immunostained with a PECAM-1 antibody 42 days after the implantation of MCF-7 tumor cells in nude mice

### DETAILED DESCRIPTION OF THE INVENTION

Anti-angiogenic therapy is an attractive approach for inhibition of tumor progression, as tumors depend upon an adequate blood supply for growth. It is disclosed herein that short peptides derived from the Aβ sequence inhibit angiogenesis, and can be used for anti-cancer therapy.

Provided are anti-angiogenic Aβ peptide fragments that can be used to treat pathological conditions mediated by undesired and/or uncontrolled or pathological angiogenesis. Provided herein is a particular anti-angiogenic motif (HHQKLVFF) which may be used in anti-tumor or anti-angiogenic therapies.

### Anti-Angiogenic Peptide Fragments

The present invention provides anti-angiogenic fragments of Aβ peptides useful for the treatment of disorders or diseases associated with pathological or unwanted angiogenesis.

The term “Aβ peptide fragment” as used herein refers to an anti-angiogenic fragment of a full length Aβ peptides (e.g., Aβ₁₋₄₀, Aβ₁₋₄₂, Aβ₁₋₄₃) and includes Aβ peptide fragment variants, homologs (such as mammalian orthologs) and isoforms, unless otherwise noted. The term also includes fragments with substitutions of one or more equivalent amino acids, or non-natural amino acids.

In one embodiment, the Aβ peptide fragment is at least one amino acid less in number than the total number of amino acids found in the full-length Aβ peptide. Full length Aβ peptides are derived from proteolytic processing of one or more isoforms of the amyloid precursor protein (APP), a transmembrane glycoprotein (Kang, J. et al. Nature (Lond.). (1987) 325: 733-736). The 39-43-amino acid-long Aβ peptide amino acid sequence begins in the ectodomain of APP and extends into the transmembrane region. Aβ is formed after sequential cleavage of APP by the β- and γ-secretases. Aβ₁₋₄₂ and Aβ₁₋₄₃ forms are specifically found in all kinds of AD plaques, indicating that those forms are critically important in AD pathology.

In a particular embodiment, the Aβ peptide fragment is at least one amino acid less in number than the total number of amino acids found in the full length Aβ₁₋₄₀ peptide. The Aβ₁₋₄₀ peptide fragment consists of, for example, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids.

In another particular embodiment, the Aβ peptide fragment is at least one amino acid less in number than the total number of amino acids found in the full length Aβ₁₋₄₂ peptide. The Aβ₁₋₄₂ fragment consists of, for example, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or 41 amino acids.

In another particular embodiment, the Aβ peptide fragment is at least one amino acid less in number than the total number of amino acids found in the full length Aβ₁₋₄₃ peptide. The Aβ₁₋₄₃ fragment consists of, for example, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, or 42 amino acids.

In one embodiment, the fragment consists of, for example, 8, 9, 10, 11, 12, 13, 14, 15, 16, 17, 18, 19, 20, 21, 22, 23, 24, 25, 26, 27, 28 or more amino acid residues, and includes the sequence HHQKLVFF.

In one embodiment, one or more of the following biologically active Aβ peptide fragments may be used to treat diseases or disorders associated with unwanted or pathological angiogenesis: the Aβ₁₋₂₈ peptide, the Aβ₁₀₋₃₅ peptide, the Aβ₁₂₋₂₈ peptide, the Aβ₁₃₋₂₀ peptide, or biologically active fragments or variants thereof.

The anti-angiogenic Aβ peptide fragment preferably contains the HHQK proteoglycan binding region, since fragments without that sequence (Aβ₂₅₋₃₅, Aβ₁₇₋₂₈, and Aβ₃₄₋₄₂) were not active, suggesting that the heparin binding motif HHQK is required to mediate the anti-angiogenic activity of Aβ. The Aβ₁₀₋₁₆ fragment was inactive even though it contains the HHQK sequence, suggesting that the HHQK proteoglycan binding motif is not sufficient to inhibit angiogenesis and that other neighboring residues are required. In particular, the LVFF sequence immediately following the HHQK domain is also required for inhibition of angiogenesis. Thus, preferred Aβ peptide fragments contain the amino acid sequence HHQKLVFF.

In one embodiment, the fragment consists of, for example, 8, 9, 10, 11, 12, 13, 14, 15, 16, 17, 18, 19, 20, 21, 22, 23, 24, 25, 26, 27, 28, 29, 30, 31, 32, 33, 34, 25, 36, 37, 38 or more amino acid residues, and includes the sequence HHQKLVFF. Such fragments may include one or more (e.g. 2, 3 or 4) substitutions of equivalent amino acids, including, e.g., non-natural amino acids.

In one embodiment, the Aβ peptide fragment is a Aβ₁₂₋₂₈ peptide containing the amino acid sequence HHQKLVFF, or a biologically active fragment or variant thereof.

In another embodiment, the Aβ peptide fragment is a Aβ₁₃₋₂₀ peptide fragment or the amino acid sequence HHQKLVFF, or a biologically active fragment or variant thereof.

In another embodiment, the Aβ peptide fragment comprises the amino acid sequence EVHHQKLVFF, or a biologically active fragment or variant thereof.

In another embodiment, the Aβ peptide fragment is, e.g., a 10, 20, 30, or 40 amino acid fragment of the Aβ peptide.

The peptide fragments are obtained, for example, by chemical synthesis, or are recombinantly produced by host cells.

Likewise, the terms variant and homologous are also used interchangeably. “Variant” or “homologous” peptide fragments will be understood to designate those containing, in relation to the native polypeptide sequence, modifications such as deletion, addition, or substitution of at least one amino acid, truncation, extension, or the addition of chimeric heterologous polypeptides. Optionally, “variant” or “homologous” peptide fragments can contain a mutation or post-translational modifications.

Among the “variant” or “homologous” polypeptides or peptide fragments, those whose amino acid sequence exhibits 80.0% to 99.9% (inclusive) identity to the native polypeptide sequence are preferred. These percentages are purely statistical and differences between two peptide sequences can be distributed randomly and over the entire sequence length.

“Variant” or “homologous” polypeptide sequences exhibiting a percentage identity with the polypeptides of the present invention can, alternatively, have 80, 81, 82, 83, 84, 85, 86, 87, 88, 89, 90, 91, 92, 93, 94, 95, 96, 97, 98, or 99 percent identity with the polypeptide sequences of the instant invention. Depending on the computer program used to calculate percent identity, the actual number of substitutions, deletions and/or insertions will vary. The expression equivalent amino acid is intended here to designate any amino acid capable of being substituted for one of the amino acids in the basic structure without, however, essentially modifying the biological activities of the corresponding peptides and as provided below.

### Amino Acid Modifications

In one embodiment, at least one non-natural amino acid is incorporated into the peptide. The terms “non-natural amino acid” and “modified amino acid” are being used interchangeably herein. In another embodiment, at least two or more, at least three or more, at least 4 or more, at least 5 or more, at least 6 or more, at least 7 or more, at least 8 or more, at least 9 or more or at least 10 or more non-natural acids are incorporated into the peptide. In other embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 non-natural amino acids are incorporated into the peptide.

Many of the D-amino acids can be incorporated in place of the natural L-amino acids, either at a specific position, or throughout the whole peptide to increase peptide stability toward proteases. Non-natural amino acids may also increase in vivo half life time and potency of peptides (see Tian, et al. (2006) Bioorg. Med. Chem. Lett., 16:1721-1725).

In some embodiment, one or more of these non-natural amino acids is a D amino acid. In other embodiments, at least two or more of these non-natural amino acids is a D amino acid. In certain embodiments, at least 3, at least 4, at least 5, at least 6, at least 7, at least 8, at least 9 or at least 10 or more of these non-natural amino acids is a D amino acid.

In one embodiment, the incorporated non-natural amino acid is selected from:

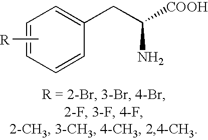

In certain other embodiments, the non-natural amino acid used for incorporation into the peptide is selected from the group consisting of 3,4-Dehydro-DL-proline; 5-Benzyloxy-DL-tryptophan; D-Alanyl-D-alanine; D-Alanyl-L-leucine; D-Arginine Hydrochloride; D-Asparagine; D-Asparagine, Monohydrate; D-Cystine; D-methionine; D-tryptophan; D-phenylalanine; DL-Alanyl-DL-leucine; DL-Alanyl-DL-leucylglycine; DL-Alanyl-DL-phenylalanine; DL-Arginine Hydrochloride; DL-Cysteine; DL-Cysteine Hydrochloride; DL-Cysteine Hydrochloride Monohydrate; DL-Histidine Hydrochloride, Monohydrate; N-Acetyl-D-leucine; N-Benzoyl-DL-methionine; N-Benzoyl-L-phenylalanine; N-Carbamyl-DL-alanine; N-Chloroacetyl-DL-phenylalanine; N-Chloroacetyl-DL-valine; O-Benzyl-D-serine; O-Benzyl-DL-serine; 3-iodo-L-tyrosine (IY) and p-benzoyl-L-phenylalanine (pBpa).

The non-natural amino acid can be incorporated into the peptide using techniques known in the art. For example, the amino acid can be incorporated during synthesis in a biological system by growing an expression system (such as a bacterial system) in media containing the non-natural amino acids. The amino acids can also be incorporated by manipulation of the genetic code of the biological system (as described, for example, in Hodgson D R, and Sanderson J M. (2004) Chem Soc Rev. 33:422-30; Hendrickson, et al. (2004) Annual Review of Biochemistry Vol. 73:147-176; Hohsaka and Sisido (2002) Curr Opin Chem Biol. 6(6):809-15; Hohsaka, et al. (2001) Biochemistry 40:11060-11064). The amino acid may also be incorporated in vitro during protein synthesis (for e.g. see Hohsaka, et al. (1999) J. Am. Chem. Soc., 121:34-40).

In certain embodiments, synthetic amino acids can be used that are designed to ensure certain two or three dimensional conformations of the peptide. In certain instances, the synthetic amino acid forms a dimer, binding at least two portions of the peptide together. In certain embodiments, the incorporation of at least one synthetic amino acid promotes formation of one or more beta sheets in the peptide. Beta-sheets are ribbon-like structures that are widespread in proteins and have the capacity to interact by means of unsatisfied hydrogen-bonding valences along their edges. In one embodiment, the synthetic amino acid mimics beta strands. In another embodiment, the synthetic amino acid blocks beta-sheet dimerization of proteins. In an alternate embodiment, the synthetic amino acid promotes dimerization of proteins. In a further embodiment, the synthetic amino acid blocks protein-protein beta-sheet interactions. In an alternate embodiment, the synthetic amino acid interacts with more than one peptide by beta-sheet formation. As a non-limiting example, in one embodiment, at least one synthetic amino acid is incorporated in at least one position of the peptide to ensure binding of beta sheets. In specific embodiments, the peptide will form at least one beta sheet. In other embodiments, the synthetic amino acid binds more than one peptide together. Certain design parameters for linear peptides to fold into particular conformations is described, for example, in Cheng (2004) Curr Opin Struct Biol. 14(4):512-20; Martinek and Fulop (2003) Eur J Biochem. 2003 270(18):3657-66; and Lacroix, et al. (1999) Curr Opin Struct Biol. 9(4):487-93.

In one embodiment, the synthetic amino acid is Hao (also Orn(1-PrCO-Hao) from hydrazine, 5-amino-2-methoxybenzoic acid and oxalic acid).

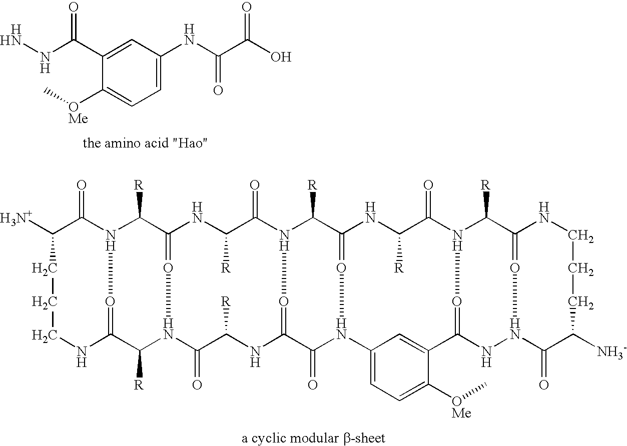

(see also PCT Publication WO 01/14412.

In other embodiments, the synthetic amino acid is L-2-aminohexanoic acid (Ahx). In yet other embodiments, the synthetic amino acid is selected from 3-iodo-L-tyrosine, ethylenediaminetetraacetic acid (EDTA)-derivatized tryptophan (Trp), 7-azatryptophan (7AW) and 5-hydroxytryptophan (SHW).

Introduction of novel functionality to peptides and proteins using transport-carrier molecules that are recognized by endogenous cellular-transport systems in the GI tract might represent one strategy for increasing intestinal absorption of peptides and proteins. In fact, this is a method that has been undertaken by numerous investigators and companies to achieve improved bioavailability. However, no such systems are available commercially as yet. The associated transport mechanisms are membrane transporters and receptor-mediated endocytosis, recognizing and internalizing specific ligands attached to macromolecules. In some embodiments, the peptide fragment is attached to a dipeptide that is recognized by a peptide-influx transporter, such as described in Han, H. K. and Amidon, G. L. (2000) Targeted prodrug design to optimize drug delivery. AAPS Pharm Sci 2:E6. In other embodiments, receptor-recognizable ligands, such as lectins, toxins, viral haemagglutinins, invasins, transferrin, and vitamins (Vitamin B12 [VB12], folate, riboflavin and biotin), can be tethered to the peptide fragment as described in Russell-Jones, G. J. (2004) Use of targeting agents to increase uptake and localization of drugs to the intestinal epithelium. J. Drug Target. 12:113-123; Hwa Kim, S. (2005) Folate receptor mediated intracellular protein delivery using PLL-PEG-FOL conjugate. J. Control. Release 103:625-634; and Lim, C. J. and Shen, W. C. (2005) Comparison of monomeric and oligomeric transferrin as potential carrier in oral delivery of protein drugs. J. Control. Release 106:273-286.

A class of short peptides, such as TAT (48-60), penetratin and oligoarginine, have been used to internalize different bioactive compounds into cells (Trehin, R. and Merkle, H. P. (2004) Chances and pitfalls of cell penetrating peptides for cellular drug delivery. Eur. J. Pharm. Biopharm. 58:209-223; Zorko, M. and Langel, U. (2005) Cell-penetrating peptides: mechanism and kinetics of cargo delivery. Adv. Drug Deliv. Rev. 57:529-545). These peptides can generally hybridize with target materials. In certain embodiments, the peptide fragment is linked to this type of short peptide to facilitate targeting.

In certain embodiments, the peptides can be stabilized by incorporation of sterically hindered non-natural amino acids, e.g. C^{α,α}-disubstituted amino acids. In a particular embodiment, peptides include incorporation of α-Trifluoromethyl substituted amino acids.

The peptide may contain modifications to the C- and/or N-terminus which include, but are not limited to amidation or acetylation. In certain embodiments, the amino acid residues contain reactive side chains, for example carboxy side chain in glutamic acid, that can be capped by capping groups known in the art. Acetylation is known to regulate many diverse protein functions, including DNA recognition, protein protein interaction and protein stability. Acetylation refers to the introduction of a COCH₃ group either at the amino terminus or on the side chain(s) of at least one lysine in the peptide(s) or peptide fragment(s). Importantly, acetylation can regulate protein stability Analysis of in vivo acetylated E2F1 shows that the acetylated version has a longer half-life (Martinez-Balbás et al., (2000) EMBO J. 19(4):662-71; see also Takemura et al. (1992) J Cell Sci. 103 (Pt 4):953-64). In certain embodiments, the amino-terminal of the peptide fragment is modified by acetylation. In certain embodiments, a lysine side chain in the peptide fragment is modified. In yet other embodiments, the peptide fragment is acetylated both at the amino terminus and on a lysine side chain.

In certain embodiments, a statine (3S,4S-4-amino-3-hydroxy-6-methylheptanoic acid) or AHPPA (3S,4S-4-amino-3-hydroxy-5-phenylpentanoic acid) residue can be substituted in place of any two amino acids of the peptide. The unusual amino acid statine has become a prototypical hydroxymethylene isostere, and is contained in pepstatin, the naturally occurring peptide produced by various Streptomyces species. It has been found that certain statine-based peptidomimetics show inhibitory activities to the β-secretases (see for e.g. Bridges, et al. (2006) Peptides 27(7):1877-85; Marcinkeviciene (2001) J. Biol. Chem. 276:23790-23794). Certain peptidomimetic p-secretase inhibitors, GL189 (H-Glu-Val-Asn-Statine-Val-Ala-Glu-Phe-NH) and P10-P4′statV (H-Lys-Thr-Glu-Glu-Ile-Ser-Glu-Val-Asn-Stat-Val-Ala-Glu-Phe-OH (Stat=(3S,4S)-Statine)), are substrate analogue BACE inhibitors. GL189 completely blocks the proteolytic activity (at 5 uM) of β-secretase in solubilized membrane fractions from BACE transfected MDCK cells, and P10-P4′statV is a potent inhibitor of APP protein (IC₅₀=30 nM).

In certain other embodiments, peptide backbone modifications can be made to the peptide fragment. These modifications can include an N-methyl, ketomethylene, hydroxyethylene, (E)-ethylene, reduced amide, ether or carba modification.

Several substitutions could be made to HHQKLVFF (motif) region of Aβ while potentially retaining the substitution antiangiogenic properties of the peptide. Specifically, the following expression indicates such equivalent substitutions for HHQKLVFF:

[RH]-H-[NQ]-[RK]-[ILV]-[ILV]-F-F

Exemplary sequences with such motifs are listed in Table 1. Sources are noted if a particular peptide sequence is a part of a naturally occurring protein.

TABLE 1

| | Source, if |
|---|---|
| Amino acid | naturally- |
| sequence | occurring |
| HHQKLVFF | human APP/Aβ |
| RHQKLVFF | rat/mouse APP/Aβ |
| HHNKLVFF | |
| RHNKLVFF | |
| HHQRLVFF | |
| RHQRLVFF | |
| HHNRLVFF | |
| RHNRLVFF | |
| HHQKIVFF | |
| RHQKIVFF | |
| HHNKIVFF | |
| RHNKIVFF | |
| HHQRIVFF | |
| RHQRIVFF | tr: Q3YB12_BACST |
| | putative regulator |
| | [Geobacillus |
| | stearothermophilus] |
| HHNRIVFF | |
| RHNRIVFF | |
| HHQKVVFF | |
| RHQKVVFF | |
| HHNKVVFF | |
| RHNKVVFF | |
| HHQRVVFF | [Q6CETO] Yarrowia |
| | lipolytica |
| | chromosome B of |
| | strain CLIB99 of |
| | Yarrowia lipolytica |
| | (trembl). |
| RHQRVVFF | |
| HHNRVVFF | |
| RHNRVVFF | |
| HHQKLIFF | |
| RHQKLIFF | |
| HHNKLIFF | |
| RHNKLIFF | |
| HHQRLIFF | |
| RHQRLIFF | |
| HHNRLIFF | |
| RHNRLIFF | |
| HHQKIIFF | |
| RHQKIIFF | |
| HHNKIIFF | |
| RHNKIIFF | |
| HHQRIIFF | |
| RHQRIIFF | |
| HHNRIIFF | |
| RHNRIIFF | |
| HHQKVIFF | |
| RHQKVIFF | |
| HHNKVIFF | |
| RHNKVIFF | |
| HHQRVIFF | |
| RHQRVIFF | |
| HHQKLLFF | |
| RHQKLLFF | |
| HHNKLLFF | |
| RHNKLLFF | |
| HHQRLLFF | |
| RHQRLLFF | |
| HHNRLLFF | |
| RHNRLLFF | Trembl sequence |
| | entry |
| | tr: Q7QS20_GIALA |
| HHQKILFF | |
| RHQKILFF | |
| HHNKILFF | |
| RHNKILFF | |
| HHQRILFF | |
| RHQRILFF | |
| HHNRILFF | |
| RHNRILFF | |
| HHQKVLFF | |
| RHQKVLFF | |
| HHNKVLFF | |
| RHNKVLFF | |
| HHQRVLFF | |
| RHQRVLFF | |
| HHNRVIFF | |
| RHNRVIFF | |

The motif search from http://motif.genome.jp/MOTIF2.html was used to search the peptide combinations in the NR-AA Trembl/Swissprot database. The substitution of physico-chemical equivalent amino acids in peptide sequences is known in the art. (Eisenberg, et al. 1984 “Amino acid scale: Normalized consensus hydrophobicity scale.” J. Mol. Biol. 179:125-142; and Mathura, et al. 2001, “New quantitative descriptors for amino acids based on multidimensional scaling of a large number of physical-chemical properties”, J. Mol. Modeling 7:445-453).

In one embodiment, the Aβ peptide fragment consists of or comprises one of the peptide sequences listed in Table 1, with optional equivalent amino acid substitutions.

The subject invention also provides biologically active peptide fragments capable of eliciting an immune response. The immune response can provide components (either antibodies or components of the cellular immune response (e.g., B-cells, helper, cytotoxic, and/or suppressor T-cells) reactive with the peptide fragment.

Fragments, as described herein, can be obtained by cleaving a polypeptide with a proteolytic enzyme (such as trypsin, chymotrypsin, or collagenase) or with a chemical reagent, such as cyanogen bromide (CNBr). Alternatively, polypeptide fragments can be generated in a highly acidic environment, for example at pH 2.5. Such polypeptide fragments may be also prepared by chemical synthesis or using hosts transformed with an expression vector containing nucleic acids encoding polypeptide fragments. The transformed host cells contain a nucleic acid and are cultured according to well-known methods; thus, expression of these fragments is possible, under the control of appropriate elements for regulation and/or expression.

The peptides can be modified by variation in the splicing of transcriptional products of the Aβ gene, genetic recombination, or by chemical synthesis. Such peptides can contain at least one modification in relation to the polypeptide sequence being modified. These modifications can include the addition, substitution, deletion of amino acids contained within the polypeptides.

Conservative substitutions whereby an amino acid of one class is replaced with another amino acid of the same type fall within the scope of the subject invention so long as the substitution does not materially alter the biological activity of the polypeptide. For example, the class of nonpolar amino acids include Ala, Val, Leu, Ile, Pro, Met, Phe, Gly and Trp; the class of uncharged polar amino acids include Ser, Thr, Cys, Tyr, Asn, and Gln; the class of acidic amino acids includes Asp and Glu; and the class of basic amino acids includes Lys, Arg, and His. In some instances, non-conservative substitutions can be made where these substitutions do not significantly detract from the biological activity of the polypeptide.

In order to extend the life of the polypeptides provided, it may be advantageous to use non-natural amino acids, for example in the D form, or alternatively amino acid analogs, such as sulfur-containing forms of amino acids. Alternative means for increasing the life of polypeptides can also be used. For example, peptide fragments can be recombinantly modified to include elements that increase the plasma, or serum half-life. These elements include, and are not limited to, antibody constant regions (see for example, U.S. Pat. No. 5,565,335, hereby incorporated by reference in its entirety, including all references cited therein), or other elements such as those disclosed in U.S. Pat. No. 6,319,691; 6,277,375; or 5,643,570, each of which is incorporated by reference in its entirety, including all references cited within each respective patent. Alternatively, the polynucleotides and genes can be recombinantly fused to elements that are useful in the preparation of immunogenic constructs for the purposes of vaccine formulation or elements useful for the isolation of the polypeptides provided.

### Linkers/Other Modifications

The peptide fragments disclosed may further contain linkers that facilitate the attachment of the fragments to a carrier molecule for delivery or diagnostic purposes. The linkers can also be used to attach fragments to solid support matrices for use in affinity purification protocols. In one embodiment, the linkers specifically exclude where the fragment is a subsequence of another peptide, polypeptide, or protein as identified in a search of protein sequence databases. In other words, the non-identical portions of the other peptide, polypeptide, of protein is not considered to be a “linker” in this aspect. Non-limiting examples of “linkers” suitable for the practice of the invention include chemical linkers (such as those sold by Pierce, Rockford, Ill.), peptides that allow for the connection of the immunogenic fragment to a carrier molecule (see, for example, linkers disclosed in U.S. Pat. Nos. 6,121,424; 5,843,464; 5,750,352; and 5,990,275, hereby incorporated by reference in their entirety). In various embodiments, the linkers can be up to 50 amino acids in length, up to 40 amino acids in length, up to 30 amino acids in length, up to 20 amino acids in length, up to 10 amino acids in length, or up to 5 amino acids in length.

The peptides of the present invention can also be coupled with other soluble polymers that are targetable carriers. Such polymers can include polyvinyl-pyrrolidone, pyran copolymer, polyhydroxypropylmethacryl-amidephenol, polyhydroxy-ethylaspartamidephenol, or polyethyl-eneoxidepolylysine substituted with palmitoyl residues. Furthermore, the peptide fragments can be coupled (preferably via a covalent linkage) to a class of biodegradable polymers useful in achieving controlled release, for example, polyethylene glycol (PEG), polylactic acid, polyepsilon caprolactone, polyhydroxy butyric acid, polyorthoesters, polyacetals, polydihydro-pyrans, polycyanoacrylates and cross-linked or amphipathic block copolymers of hydrogels. Cholesterol and similar molecules can be linked to the peptide fragments to increase and prolong bioavailability.

In other specific embodiments, the peptides may be expressed as a fusion, or chimeric protein product (joined via a peptide bond to a heterologous protein sequence (e.g., a different protein)). Such a chimeric product can be made by ligating the appropriate nucleic acid sequences encoding the desired amino acid sequences to each other by methods known in the art, in the proper coding frame, and expressing the chimeric product by methods commonly known in the art (see, for example, U.S. Pat. No. 6,342,362, hereby incorporated by reference in its entirety; Altendorf, et al. 1999-WWW, 2000 “Structure and Function of the Fₒ Complex of the ATP Synthase from Escherichia Coli,” J. of Experimental Biology 203:19-28, G. B.; Baneyx 1999 Biotechnology 10:411-21; Eihauer, et al. 2001 J. Biochem. Biophys. Methods 49:455-65; Jones, et al. 1995 J. Chromatography 707:3-22; Jones, et al. 1995 J. of Chromatography A. 707:3-22; Margolin, et al. 2000 Methods 20:62-72; Puig, et al. 2001 Methods 24:218-29; Sassenfeld, et al. 1990 Tib. Tech. 8:88-93; Sheibani, et al. 1999 Prep. Biochem. & Biotechnol. 29(1):77-90; Skerra, et al. 1999 Biomolecular Engineering 16:79-86; Smith, et al. 1998 The Scientist 12(22):20; Smyth, et al. 2000 Methods in Molecular Biology, 139:49-57; Unger, et al. 1997 The Scientist 11(17):20; each of which is hereby incorporated by reference in their entireties). Alternatively, such a chimeric product may be made by protein synthetic techniques, e.g., by use of a peptide synthesizer. Fusion peptides can comprise polypeptides and one or more protein transduction domains, as described above. Such fusion peptides are particularly useful for delivering the cargo polypeptide through the cell membrane.

The peptide fragments can be administered directly (e.g., alone or in a liposomal formulation or complexed to a carrier, e.g. PEG)) (see for example, U.S. Pat. Nos. 6,147,204 and 6,011,020).

Therefore, in one embodiment, the peptide fragments can be attached to a non-immunogenic, high molecular weight compound such as polyethylene glycol (PEG) or other water soluble pharmaceutically acceptable polymer as described herein. In one embodiment, the compound is associated with the PEG molecule through covalent bonds. Where covalent attachment is employed, PEG may be covalently bound to a variety of positions on the peptide.

In another embodiment, the fragment is bonded to a 5′-thiol through a maleimide or vinyl sulfone functionality. In one embodiment, a plurality of peptide fragments can be associated with a single PEG molecule. The fragments can be the same or different sequences and modifications. In yet a further embodiment, a plurality of PEG molecules can be attached to each other. In this embodiment, one or more peptide fragments to the same target or different targets can be associated with each PEG molecule. In embodiments where fragments specific for the same target are attached to PEG, there is the possibility of bringing the same targets in close proximity to each other in order to generate specific interactions between the same targets. Where multiple fragments specific for different targets are attached to PEG, there is the possibility of bringing the distinct targets in close proximity to each other in order to generate specific interactions between the targets. In addition, in embodiments where there are peptide fragments to the same target or different targets associated with PEG, another drug can also be associated with PEG. Thus the complex would provide targeted delivery of the drug, with PEG serving as a Linker.

Attempts have also been made to impart site targetability to the PEG-modified carrier in order to reduce the side effects and improve drug efficacy. An example of such attempt is the modification of a PEG-modified carrier, which can be for peptide fragments, further with a PEG-modified antibody (Maruyama K. et al. (1995) Biochim. Biophys. Acta, 1234:74).

In one embodiment, the PEG modification is through the use of a chemically modified PEG such as described in U.S. Patent Publication No. 2005/0277586. In some embodiments, the modified PEG is attached to a peptide carrier that binds to the Abeta peptide. In other embodiments, the PEG is attached to a peptide carrier that is linked to the Aβ peptide fragment during or after production of the fragment. In certain instances, the Aβ fragment and an additional peptide linker are encoded in a plasmid that is expressed to produce a chimeric Aβ fragment.

To maximize the pharmacological benefits of PEGylation, a stable bond is formed between the PEG polymer and peptide fragment of choice. In general, a PEG polymer is first chemically activated in order to react with a peptide fragment. The activated PEG derivative is then covalently linked to a reactive group on the peptide fragment. Changes in the size, structure, and molecular weight of PEG polymers can affect the biological activity of the attached fragment. In general, PEGylation of a polypeptide lowers its renal clearance, increases its half-life, and improves its biological activity. An important aspect of PEGylation is the incorporation of various PEG functional groups that are used to attach the PEG to the peptide or protein. Chemical modifications and requirements for PEGylation of peptides and proteins are reviewed in Roberts, et al. (2002) Adv. Drug Deliv. Rev. 54:459-476.

Advanced PEGylation can also be used to create prodrugs, where active fragments are released by degradation of more complex molecules (prodrugs) under physiological conditions, providing a powerful method of drug delivery. Site-specific PEGylation, such as, for example, coupling PEG reagents to protein thiol groups on cysteine can offer advantages in that cysteines are typically less abundant in proteins than other polymer attachment sites, such as amino groups, resulting in more selective PEGylation of the target protein. In addition to minimizing loss of biological activity, site-specific PEGylation can also reduce immunogenicity. Thiol groups may be naturally occurring or the biomolecule may be modified or engineered to contain a thiol suitable for conjugation.

In one embodiment, the PEG is linked to a peptide through activating the polymer for the conjugation using, for example, PEG-Met-Nle-OSu. In other embodiments, the PEG is linked through a linker. The PEG can be any commercially available PEG. In certain embodiments, the PEG for conjugation is selected from the following commercially available PEG molecules:

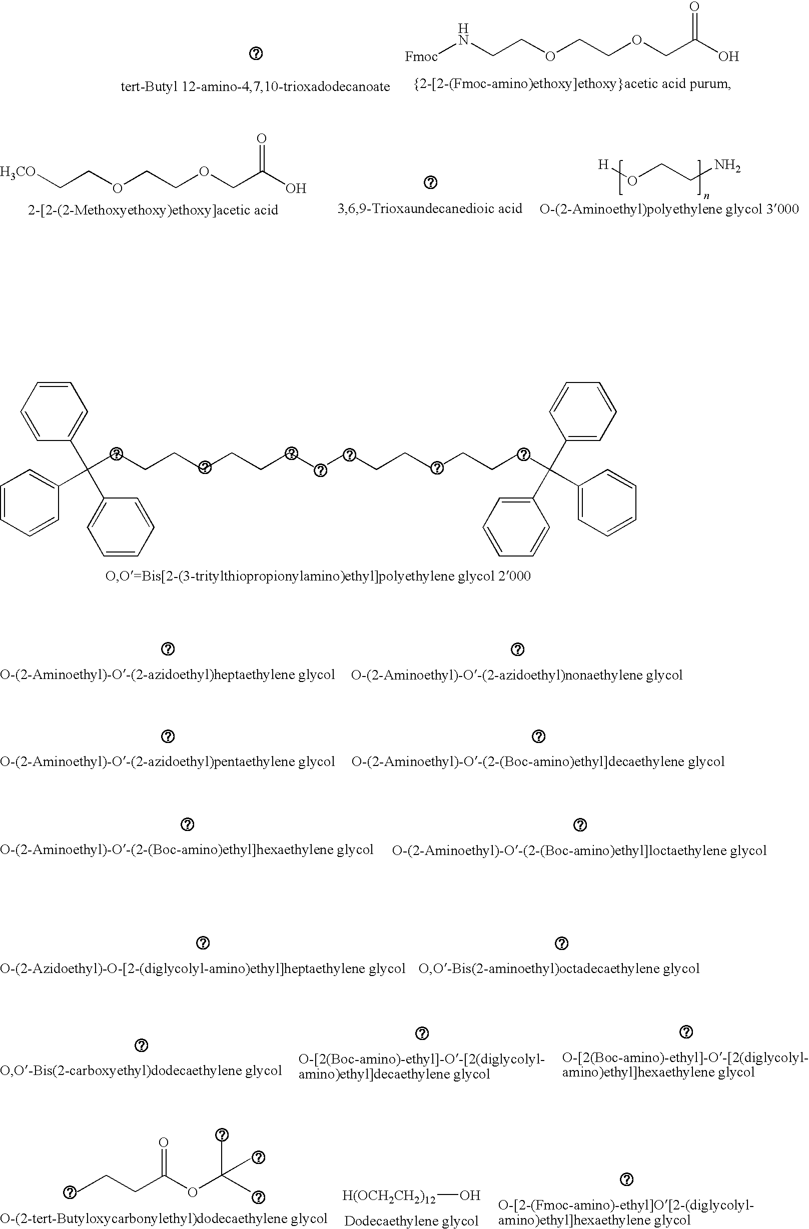

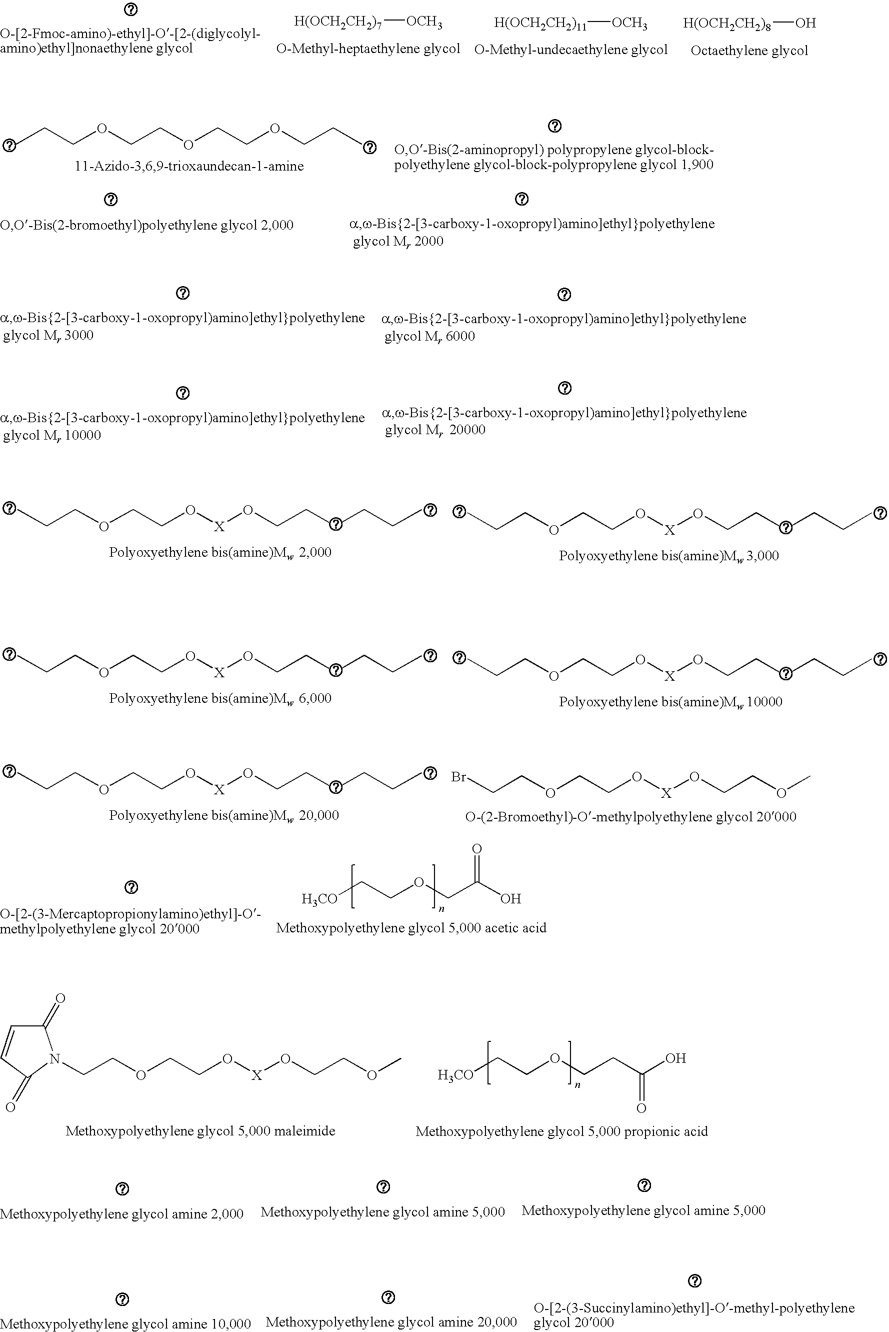

O,O′-Bis[2-(N-Succinimidyl-succinylamino)ethyl]polyethylene glycol 3′000
Poly(ethylene glycol) diacid 600
Polyethylene glycol dimesylate 2,000
Polyethylene glycol dimesylate purum, 4,000
O-[2-(3-Mercaptopropionylamino)ethyl]-O′-methylpolyethylene glycol 5′000
Methoxypolyethylene glycol amine hydrochloride 750
O-Methyl-O′-succinylpolyethylene glycol 2′000
O-Methyl-O′-succinylpolyethylene glycol 5′000
O-[2-(6-Oxocaproylamino)ethyl]-O′-methylpolyethylene glycol 2′000
O-[2-(6-Oxocaproylamino)ethyl]-O′-methylpolyethylene glycol 5′000
Polyethylene glycol monomethyl ether mesylate 2,000
Polyethylene glycol monomethyl ether mesylate 5,000
O-[(N-Succinimidyl)succinyl-aminoethyl]-O′-methylpolyethylene glycol 2′000
O-[(N-Succinimidyl)succinyl-aminoethyl]-O′-methylpolyethylene glycol 5′000

The peptide fragments of the invention can also include other conjugate groups covalently bound to functional groups. Conjugate groups of the invention include polyamines, polyamides, polyethylene glycols, polyethers, groups that enhance the pharmacodynamic properties of peptides, and groups that enhance the pharmacokinetic properties of peptides. Groups that enhance the pharmacodynamic properties, in the context of this invention, include groups that improve peptide bioavailability, enhance peptide resistance to degradation, and/or strengthen target interactions.

Lipidization, which is the covalent conjugation of a hydrophobic moiety or the noncovalent interaction with a hydrophobic compound, can increase the lipophilicity of peptide and protein molecules (Hashimoto, M. et al. (1989) Synthesis of palmitoyl derivatives of insulin and their biological activities. Pharm. Res. 6:171-176; and Goldberg, M. and Gomez-Orellana, I. (2003) Challenges for the oral delivery of macromolecules. Nat. Rev. Drug Discov. 2:289-295) whereas conjugation with polyethylene glycol (PEG) improves solubility and offers protection from enzymatic degradation (Calceti, P. et al. (2004) Development and in vivo evaluation of an oral insulin-PEG delivery system. Eur. J. Pharm. Sci. 22: 315-323; and Basu, A. et al. (2006) Structure-function engineering of interferon-beta-1b for improving stability, solubility, potency, immunogenicity, and pharmacokinetic properties by site-selective mono-PEGylation. Bioconjugate Chem. 17: 618-630).

CNS penetration is favored by low molecular weight, lack of ionization at physiological pH, and lipophilicity. In one particular embodiment, the peptide fragment is designed as a lipophilic precursor. In one embodiment, the fragment is in immunoliposomes (antibody-directed liposome). In certain embodiments, gangliosides or PEG-derivatized lipids are inserted within the bilayer of conventional liposomes, as these modifications prolong considerably the liposome half-life in the circulation. Liposomes coated with the inert and biocompatible polymer PEG are widely used and are often referred to as “sterically stabilized” or “stealth liposomes”. PEG coating is believed to prevent recognition of liposomes by macrophages due to reduced binding of plasma proteins. In certain embodiments, a cell-specific ligand is attached to the distal end of a few lipid-conjugated PEG molecules rather than conjugated to a lipid head group on the surface of a PEG-conjugated liposome.

Carrier-mediated transport (CMT) and receptor-mediated transport (RMT) pathways are available for certain circulating nutrients or peptides. Several transport systems for nutrients and endogenous compounds are present that can target a peptide fragment to the brain. These include (a) the hexose transport system for glucose and mannose, (b) the neutral amino acid transport system for phenylalanine, leucine and other neutral amino acids, (c) the acidic amino acid transport system for glutamate and aspartate, (d) the basic amino acid transport system for arginine and lysine, (e) the b-amino acid transport system for b-alanine and taurine, (f) the monocarboxylic acid transport system for lactate and short-chain fatty acids such as acetate and propionate, (g) the choline transport system for choline and thiamine, (h) the amine transport system for mepyramine, (i) the nucleoside transport system for purine bases such as adenine and guanine, but not pyrimidine bases, and (j) the peptide transport system for small peptides such as enkephalins, thyrotropin-releasing hormone, argininevasopressin etc. Utilization of differences in the affinity and the maximal transport activity among these transport systems expressed at the BBB is an attractive strategy for controlling the delivery and retention of peptide fragments into the brain.

Receptor-mediated delivery to the brain employs chimeric peptide technology, wherein a non-transportable peptide fragment is conjugated to a transport vector which is a modified protein or receptor-specific monoclonal antibody that undergoes receptor-mediated transcytosis through the BBB in-vivo. Conjugation of the fragment(s) to a transport vector is facilitated with chemical linkers, avidin-biotin technology, polyethylene glycol linkers, or liposomes. Multiple classes of therapeutics have been delivered to the brain with the chimeric peptide technology, including peptide-based pharmaceuticals such as a vasoactive peptide analog or neurotrophins such as brain-derived neurotrophic factor. In certain embodiments, it is desirable to attach the peptide fragment to the transport vector via a cleavable disulfide linkage that ensures the fragment is still pharmacologically active following release from the transport vector owing to cleavage of the disulfide bond. Depending on the chemistry of the disulfide linker, a molecular adduct will remain attached to the fragment following disulfide cleavage, and the molecular adduct must not interfere with fragment activity (see for e.g. Oldendorf, W. H. (1970) Measurement of brain uptake of radiolabele substances using a tritiated water internal standard. Brain Res, 24:1629-1639; Pardridge, W. M., et al. (1990) Comparison of in-vitro and in-vivo models of drug transcytosis through blood-brain barrier. J Pharm Exp Ther, 253:884-891; Levin, V. A. (1980) Relationship of octanol/water partition coefficient and molecular weight to rat brain capillary permeability. J Med Chem, 23:682-684).

Increasing the amount of Aβ peptide fragment activity within a tissue is useful in treating a variety of angiogenic diseases, such as cancers, tumors, and/or malignancies. Thus, according to the methods provided, the amount of Aβ peptide fragment activity can be increased within a tissue by directly administering the Aβ peptide fragment to a patient suffering from an angiogenic disease (such as exogenous delivery of the Aβ peptide fragment) or by indirect or genetic means (such as delivery of a polynucleotide encoding the Aβ peptide fragment or upregulating the endogenous Aβ peptide fragment activity). Non-limiting examples of such cancers, tumors, and/or malignancies that can be treated using the methods of the invention include prostate cancer, breast cancer, melanoma, chronic myelogenous leukemia, cervical cancer, adenocarcinomas, lymphoblastic leukemia, colorectal cancer, and lung carcinoma.

The peptide fragments or nucleic acids encoding them can be used in screening, or aiding in the diagnosis of, an individual suspected of having an angiogenic or angiogenesis-mediated disease. The peptide fragments disclosed herein and nucleic acids encoding them can be used to detect the Aβ peptide in hybridization assays by the use of complementary sequences. The presence of a significantly increased amount of Aβ peptide fragment is associated with an indication of Alzheimer's disease. The presence of a significantly decreased amount of Aβ peptide is associated with an indication of an angiogenic disease, such as a malignancy or cancer. Aβ gene product can be detected by well-known methodologies including, and not limited to, Western blots, enzyme linked immunoassays (ELISAs), radioimmunoassays (RIAs), Northern blots, Southern blots, PCR-based assays, or other assays for the quantification of gene product known to the skilled artisan. This information, in conjunction with other information available to the skilled practitioner, assists in making a diagnosis.

In one aspect, the subject invention concerns a method of inhibiting angiogenesis in a patient in need of anti-angiogenesis therapy by administration of biologically active Aβ peptide fragment to the patient.

In one embodiment, a treatment for a pathological condition selected from the group consisting of cancer, arthritis, atherosclerosis, psoriasis, macular degeneration, and diabetic retinopathy by administering to the patient a therapeutically effective amount of an Aβ peptide fragment.

In one embodiment, biologically active variants of the Aβ peptide fragments are utilized, wherein the variants have a substitution at the 21 amino acid position, or the 22 amino acid position, or 23 amino acid position, or combinations thereof. In a specific embodiment, the substitution(s) is a conservative substitution which does not materially alter the biological activity of the polypeptide.

Various means for delivering polypeptides to a cell can be utilized to carry out the methods provided. For example, protein transduction domains (PTDs) can be fused to the polypeptide, producing a fusion polypeptide, in which the PTDs are capable of transducing the polypeptide cargo across the plasma membrane (Wadia, J. S. and Dowdy, S. F., Curr. Opin. Biotechnol. 2002, 13(1), 52-56). Examples of PTDs include the Drosophila homeotic transcription protein antennapedia (Antp), the herpes simples virus structural protein VP22, and the human immuno-deficiency virus 1 (HIV-1) transcriptional activator Tat protein.

According to the method of angiogenesis inhibition provided, recombinant cells can be administered to a patient, wherein the recombinant cells have been genetically modified to express Aβ peptide fragments disclosed herein.

The method of angiogenesis inhibition provided can be used to treat a patient suffering from cancer, or as a cancer preventative. The method of tumor inhibition provided can be used to treat patients suffering from a variety of cancers including, but not limited, to cancer of the breast, prostate, melanoma, chronic myelogenous leukemia, cervical cancer, adenocarcinoma, lymphoblastic leukemia, colorectal cancer, and lung carcinoma. According to the methods provided, various other anti-cancer or anti-tumor compounds, such as cytotoxic agents, can be administered in conjunction with Aβ peptide fragments.

### Nucleotide Sequences Encoding Aβ Fragments

In another aspect, the subject invention provides isolated and/or purified nucleotide sequences comprising a polynucleotide sequence encoding the amino acid sequence of the peptide fragments disclosed herein.

Also provided are isolated nucleic acid molecules comprising polynucleotides encoding the Aβ peptide fragments. One aspect of the invention provides isolated nucleic acid molecules comprising polynucleotides having a nucleotide sequence selected from the group consisting of: (a) a nucleotide sequence encoding any of the amino acid sequences of the polypeptides described herein including in Table 1; and (b) a nucleotide sequence complementary to any of the nucleotide sequences in (a).

Further embodiments of the invention include isolated nucleic acid molecules that comprise a polynucleotide having a nucleotide sequence at least 90% identical, and more preferably at least 95%, 96%, 97%, 98% or 99% identical to any of the nucleotide sequences in (a) or (b) above.

Nucleotide, polynucleotide, or nucleic acid sequences(s) are understood to mean, according to the present invention, either a double-stranded DNA, a single-stranded DNA, or products of transcription of the said DNAs (e.g., RNA molecules). The nucleic acid, polynucleotide, or nucleotide sequences can be isolated, purified (or partially purified), by separation methods including, but not limited to, ion-exchange chromatography, molecular size exclusion chromatography, affinity chromatography, or by genetic engineering methods such as amplification, cloning or subcloning.

Optionally, the polynucleotide sequences can also contain one or more polynucleotides encoding heterologous polypeptide sequences (e.g., tags that facilitate purification of the polypeptides of the invention (see, for example, U.S. Pat. No. 6,342,362, hereby incorporated by reference in its entirety; Altendorf, et al. 1999-WWW, 2000 “Structure and Function of the Fₒ Complex of the ATP Synthase from Escherichia Coli,” J. of Experimental Biology 203:19-28, G. B.; Baneyx 1999 Biotechnology 10:411-21; Eihauer, et al. 2001 J. Biochem. Biophys. Methods 49:455-65; Jones, et al. 1995 J. Chromatography 707:3-22; Jones, et al. 1995 J. of Chromatography A. 707:3-22; Margolin, et al. 2000 Methods 20:62-72; Puig, et al. 2001 Methods 24:218-29; Sassenfeld, et al. 1990 Tib. Tech. 8:88-93; Sheibani, et al. 1999 Prep. Biochem. & Biotechnol. 29(1):77-90; Skerra, et al. 1999 Biomolecular Engineering 16:79-86; Smith, et al. 1998 The Scientist 12(22):20; Smyth, et al. 2000 Methods in Molecular Biology, 139:49-57; Unger, et al. 1997 The Scientist 11(17):20; each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or INVITROGEN (San Diego, Calif.).

### Vectors

Other aspects provide vectors containing one or more of the polynucleotides provided, such as vectors containing nucleotides encoding biologically active Aβ peptide fragments. The vectors can be vaccine, replication, or amplification vectors. In some embodiments, the polynucleotides are operably associated with regulatory elements capable of causing the expression of the polynucleotide sequences. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations of the aforementioned vector sources, such as those derived from plasmid and bacteriophage genetic elements (e.g., cosmids and phagemids).

As indicated above, vectors can also comprise elements necessary to provide for the expression and/or the secretion of a polypeptide, such as a fragment of the Aβ peptide, encoded by the nucleotide sequences provided in a given host cell. The vector can contain one or more elements selected from the group consisting of a promoter, signals for initiation of translation, signals for termination of translation, and appropriate regions for regulation of transcription. In certain embodiments, the vectors can be stably maintained in the host cell and can, optionally, contain signal sequences directing the secretion of translated protein. Other embodiments provide vectors that are not stable in transformed host cells. Vectors can integrate into the host genome or be autonomously-replicating vectors.

In a specific embodiment, a vector comprises a promoter operably linked to a protein or peptide-encoding nucleic acid sequence, one or more origins of replication, and, optionally, one or more selectable markers (e.g., an antibiotic resistance gene). Non-limiting exemplary vectors for the expression of polypeptides include pBr-type vectors, pET-type plasmid vectors (PROMEGA), pBAD plasmid vectors (INVITROGEN) or those provided in the examples below. Furthermore, vectors are useful for transforming host cells for the cloning or expression of the nucleotide sequences provided.

Promoters which may be used to control expression include, but are not limited to, the CMV promoter, the SV40 early promoter region (Bernoist and Chambon 1981 Nature 290:304-310), the promoter contained in the 3′ long terminal repeat of Rous sarcoma virus (Yamamoto, et al. 1980 Cell 22:787-797), the herpes thymidine kinase promoter (Wagner et al. 1981 Proc. Natl. Acad. Sci. USA 78:1441-1445), the regulatory sequences of the metallothionein gene (Brinster et al. 1982 Nature 296:39-42); prokaryotic vectors containing promoters such as the β-lactamase promoter (Villa-Kamaroff, et al. 1978 Proc. Natl. Acad. Sci. USA 75:3727-3731), or the tac promoter (DeBoer, et al. 1983 Proc. Natl. Acad. Sci. USA 80:21-25); see also, “Useful Proteins from Recombinant Bacteria” in Scientific American, 1980, 242:74-94; plant expression vectors comprising the nopaline synthetase promoter region (Herrera-Estrella et al. 1983 Nature 303:209-213) or the cauliflower mosaic virus 35S RNA promoter (Gardner, et al. 1981 Nucl. Acids Res. 9:2871), and the promoter of the photosynthetic enzyme ribulose biphosphate carboxylase (Herrera-Estrella et al. 1984 Nature 310:115-120); promoter elements from yeast or fungi such as the Gal 4 promoter, the ADC (alcohol dehydrogenase) promoter, PGK (phosphoglycerol kinase) promoter, and/or the alkaline phosphatase promoter.

### Homologous Nucleotide Sequences

Provided herein are “homologous” or “modified” nucleotide sequences. Modified nucleic acid sequences will be understood to mean any nucleotide sequence obtained by mutagenesis according to techniques well known to persons skilled in the art, and exhibiting modifications in relation to the normal sequences. For example, mutations in the regulatory and/or promoter sequences for the expression of a polypeptide that result in a modification of the level of expression of a polypeptide provide for a “modified nucleotide sequence”. Likewise, substitutions, deletions, or additions of nucleic acid to the polynucleotides provide for “homologous” or “modified” nucleotide sequences. In various embodiments, “homologous” or “modified” nucleic acid sequences have substantially the same biological or serological activity as the native (naturally occurring) Aβ peptide fragments. A “homologous” or “modified” nucleotide sequence will also be understood to mean a splice variant of the polynucleotides of the instant invention or any nucleotide sequence encoding a “modified polypeptide” as defined below.

A homologous nucleotide sequence, as described herein, encompasses a nucleotide sequence having a percentage identity with the bases of the nucleotide sequences of between at least (or at least about) 80.0% to 99.9% (inclusive), or 85% to 99%, or 90% to 99%, or 95% to 99%.

In various embodiments, homologous sequences exhibiting a percentage identity with the bases of the nucleotide sequences described can have 80, 81, 82, 83, 84, 85, 86, 87, 88, 89, 90, 91, 92, 93, 94, 95, 96, 97, 98, or 99 percent identity with the polynucleotide sequences of the instant invention.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman 1988 Proc. Natl. Acad. Sci. U.S.A. 85(8):2444-2448; Altschul, et al. 1990 J. Mol. Biol. 215(3):403-410; Thompson, et al. 1994 Nucleic Acids Res. 22(2):4673-4680; Higgins, et al. 1996 Methods Enzymol. 266:383-402; Altschul, et al. 1990 J. Mol. Biol. 215(3):403-410; Altschul, et al. 1993 Nature Genetics 3:266-272).

Also provided are nucleotide sequences complementary to any of the polynucleotide sequences disclosed herein. Thus, the invention is understood to include any DNA whose nucleotides are complementary to those of the sequence of the invention, and whose orientation is reversed (e.g., an antisense sequence).

Further provided are fragments of the polynucleotide sequences disclosed herein. Representative fragments of the polynucleotide sequences will be understood to mean any nucleotide fragment having at least 8 or 9 successive nucleotides, preferably at least 12 successive nucleotides, and still more preferably at least 15 or at least 20 successive nucleotides of the sequence from which it is derived. The upper limit for such fragments is the total number of polynucleotides found in the sequence encoding for Aβ₁₋₄₂ peptide, (or, in certain embodiments, the open reading frame (ORF) identified herein). The appropriate fragments thereof encoding for a specific peptide are also useful. For example, nucleotide sequences that are Aβ peptide fragment homologs, or fragments thereof, which have been previously identified, can be utilized to carry out the method for inhibiting angiogenesis of the subject invention.

### Hybridization and Detection Probes

Among these representative fragments, those capable of hybridizing under stringent conditions with a nucleotide sequence are preferred. Conditions of high or intermediate stringency are provided infra and are chosen to allow for hybridization between two complementary DNA fragments. Hybridization conditions for a polynucleotide of about 300 bases in size will be adapted by persons skilled in the art for larger- or smaller-sized oligonucleotides, according to methods well known in the art (see, for example, Sambrook, et al. 1989 Molecular Cloning, A Laboratory Manual, Second Edition, Cold Spring Harbor Press, N.Y., pp. 9.47-9.57).

Also provided are detection probes (e.g., fragments of the disclosed polynucleotide sequences) for hybridization with a target sequence or an amplicon generated from the target sequence. Such a detection probe will advantageously have as sequence a sequence of at least 9, 12, 15, 20, 25, 30, 35, 40, 45, 50, 55, 60, 65, 70, 75, 80, 85, 90, 95, or 100 nucleotides. Alternatively, detection probes can comprise 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127 and up to, for example, 128 consecutive nucleotides of the disclosed nucleic acids. The detection probes can also be used as labeled probe or primer in the subject invention. Labeled probes or primers are labeled with a radioactive compound or with another type of label. Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications.

The nucleotide sequences disclosed may also be used in analytical systems, such as DNA chips. DNA chips and their uses are well known in the art and (see for example, U.S. Pat. Nos. 5,561,071; 5,753,439; 6,214,545; Schena, et al. 1996 BioEssays 18:427-431; Bianchi, et al. 1997 Clin. Diagn. Virol. 8:199-208; each of which is hereby incorporated by reference in their entireties) and/or are provided by commercial vendors such as AFFYMETRIX, Inc. (Santa Clara, Calif.).

Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under moderate to high stringency conditions by techniques well known in the art, as described, for example, in Keller, G. H., M. M. Manak 1987 DNA Probes, Stockton Press, New York, N.Y., pp. 169-170.

By way of example, hybridization of immobilized DNA on Southern blots with ³²P-labeled gene-specific probes can be performed by standard methods (Maniatis, et al. 1982 Molecular Cloning: A Laboratory Manual, Cold Spring Harbor Laboratory, New York). In general, hybridization and subsequent washes can be carried out under moderate to high stringency conditions that allow for detection of target sequences with homology to the exemplified polynucleotide sequence. For double-stranded DNA gene probes, hybridization can be carried out overnight at 20-25° C. below the melting temperature (Tm) of the DNA hybrid in 6×SSPE, 5×Denhardt's solution, 0.1% SDS, 0.1 mg/ml denatured DNA. The melting temperature is described by the following formula (Beltz et al. 1983 Methods of Enzymology, R. Wu, L. Grossman and K. Moldave [eds.] Academic Press, New York 100:266-285).

Tₘ=81.5° C.+16.6 Log [Na+]+0.41(% G+C)−0.61(% formamide)−600/length of duplex in base pairs.

Washes are typically carried out as follows:

(1) twice at room temperature for 15 minutes in 1×SSPE, 0.1% SDS (low stringency wash);

(2) once at Tₘ−20° C. for 15 minutes in 0.2×SSPE, 0.1% SDS (moderate stringency wash).

For oligonucleotide probes, hybridization can be carried out overnight at 10-20° C. below the melting temperature (Tₘ) of the hybrid in 6×SSPE, 5×Denhardt's solution, 0.1% SDS, 0.1 mg/ml denatured DNA. Tₘ for oligonucleotide probes can be determined by the following formula:

Tₘ (° C.)=2 (number T/A base pairs)+4 (number G/C base pairs) (Suggs et al. 1981 ICN—UCLA Symp. Dev. Biol. Using Purified Genes, D. D. Brown [ed.], Academic Press, New York, 23:683-693).

Washes can be carried out as follows:

(1) twice at room temperature for 15 minutes 1×SSPE, 0.1% SDS (low stringency wash;

2) once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used:

1 Low: 1 or 2×SSPE, room temperature Low: 1 or 2×SSPE, 42° C. Moderate: 0.2× or 1×SSPE, 65° C. High: 0.1×SSPE, 65° C.

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art (see, for example, Sambrook, et al. 1989 Molecular Cloning, A Laboratory Manual, Second Edition, Cold Spring Harbor Press, N.Y., pp. 9.47-9.57; and Ausubel, et al. 1989 Current Protocols in Molecular Biology, Green Publishing Associates and Wiley Interscience, N.Y., each incorporated herein in its entirety).

A further non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art (see, for example, Sambrook et al. 1989 Molecular Cloning, A Laboratory Manual, Second Edition, Cold Spring Harbor Press, N.Y., pp. 9.47-9.57; and Ausubel et al 1989 Current Protocols in Molecular Biology, Green Publishing Associates and Wiley Interscience, N.Y., each of which is incorporated herein in its entirety).

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

It is also well known in the art that restriction enzymes can be used to obtain functional fragments of the subject DNA sequences. For example, Bal31 exonuclease can be conveniently used for time-controlled limited digestion of DNA (commonly referred to as “erase-a-base” procedures). See, for example, Maniatis, et al. 1982 Molecular Cloning: A Laboratory Manual, Cold Spring Harbor Laboratory, New York; Wei, et al. 1983 J. Biol. Chem. 258:13006-13512. The nucleic acid sequences disclosed can also be used as molecular weight markers in nucleic acid analysis procedures.

### Host Cells

Provided are host cells transformed by a polynucleotide according to the invention and the production of Aβ peptide fragments by the transformed host cells. In some embodiments, transformed cells comprise an expression vector containing polynucleotide sequences for an Aβ peptide fragment. Other embodiments provide for host cells transformed with nucleic acids. Yet other embodiments provide transformed cells comprising an expression vector containing fragments of Aβ polynucleotide sequences. Transformed host cells can be cultured under conditions allowing the replication and/or the expression of the nucleotide sequences provided. Expressed polypeptides are recovered from culture media and purified, for further use, according to methods known in the art.

The host cell may be chosen from eukaryotic or prokaryotic systems, for example bacterial cells (Gram negative or Gram positive), yeast cells, animal cells, plant cells, and/or insect cells using baculovirus vectors. In some embodiments, the host cell for expression of the polypeptides include, and are not limited to, those taught in U.S. Pat. Nos. 6,319,691; 6,277,375; 5,643,570; 5,565,335; Unger, et al. 1997 The Scientist 11(17):20; or Smith, et al. 1998 The Scientist 12(22):20, each of which is incorporated by reference in its entirety, including all references cited within each respective patent or reference. Other exemplary, and non-limiting, host cells include Staphylococcus spp., Enterococcus spp., E. coli, and Bacillus subtilis; fungal cells, such as Streptomyces spp., Aspergillus spp., S. cerevisiae, Schizosaccharomyces pombe, Pichia pastoris, Hansela polymorpha, Kluveromyces lactis, and Yarrowia lipolytica; insect cells such as Drosophila S2 and Spodoptera Sf9 cells; animal cells such as CHO, COS, HeLa, C127, 3T3, BHK, 293 and Bowes melanoma cells; and plant cells. A great variety of expression systems can be used to produce the polypeptides provided and polynucleotides can be modified according to methods known in the art to provide optimal codon usage for expression in a particular expression system.

Furthermore, a host cell strain may be chosen that modulates the expression of the inserted sequences, modifies the gene product, and/or processes the gene product in the specific fashion. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product whereas expression in yeast will produce a glycosylated product. Expression in mammalian cells can be used to provide “native” glycosylation of a heterologous protein. Furthermore, different vector/host expression systems may effect processing reactions to different extents.

Nucleic acids and/or vectors can be introduced into host cells by well-known methods, such as, calcium phosphate transfection, DEAE-dextran mediated transfection, transfection, microinjection, cationic lipid-mediated transfection, electroporation, transduction, scrape loading, ballistic introduction and infection (see, for example, Sambrook, et al. 1989 Molecular Cloning: A Laboratory Manual, 2.sup.nd Ed., Cold Spring Harbor Laboratory Press, Cold Spring Harbor, N.Y.).

The subject invention also provides for the expression of a polypeptide, derivative, or a variant (e.g., a splice variant) encoded by a polynucleotide sequence disclosed herein. Alternatively, the invention provides for the expression of a polypeptide fragment obtained from a polypeptide, derivative, or a variant encoded by a polynucleotide fragment derived from the polynucleotide sequences disclosed herein. In either embodiment, the disclosed sequences can be regulated by a second nucleic acid sequence so that the polypeptide or fragment is expressed in a host transformed with a recombinant DNA molecule according to the subject invention. For example, expression of a protein or peptide may be controlled by any promoter/enhancer element known in the art.

The subject invention also provides nucleic acid-based methods for the identification of the presence of the Aβ gene, or fragments or variants thereof, in a sample. These methods can utilize the nucleic acids provided and are well known to those skilled in the art (see, for example, Sambrook, et al. 1989 Molecular Cloning, A Laboratory Manual, Second Edition, Cold Spring Harbor Press, N.Y., pp. 9.47-9.57, or Abbaszadega, et al. 2001 Reviews in Biology and Biotechnology, 1(2):21-26). Among the techniques useful in such methods are enzymatic gene amplification (or PCR), Southern blots, Northern blots, or other techniques utilizing nucleic acid hybridization for the identification of polynucleotide sequences in a sample. The nucleic acids can be used to screen individuals for disorders associated with dysregulation of the Aβ gene or its transcriptional products.

The subject invention also provides polypeptides encoded by nucleotide sequences of the invention. The subject invention also provides fragments of at least 5 amino acids of a polypeptide encoded by the polynucleotides of the instant invention.

### Pharmaceutical Formulations and Administration

As used herein, the term “administration” or “administering” refers to the process of delivering an agent to a patient. The process of administration can be varied, depending on the agent, or agents, and the desired effect. Administration can be accomplished by any means appropriate for the therapeutic agent, for example, by oral, parenteral, mucosal, pulmonary, topical, catheter-based, rectal, intracranial, intracerebroventricular, intracerebral, intravaginal or intrauterine delivery. Parenteral delivery can include for example, subcutaneous intravenous, intrauscular, intra-arterial, and injection into the tissue of an organ, particularly tumor tissue. Mucosal delivery can include, for example, intranasal delivery. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid. Gene therapy protocol is also considered an administration in which the therapeutic agent is a polynucleotide capable of accomplishing a therapeutic goal when expressed as a transcript or a polypeptide into the patient.

In one embodiment, the Aβ peptide fragment is administered in an effective amount to inhibit pathological angiogenesis. As used herein, the term “angiogenesis” is intended to refer to the process by which new blood vessels are formed and which is essential to a variety of normal body activities (such as reproduction, development, and wound repair). The process is believed to involve a complex interplay of molecules which both stimulate and inhibit the growth of endothelial cells, the primary cells of the capillary blood vessels. Under normal conditions, these molecules appear to maintain the microvasculature in a quiescent state (i.e., one of no capillary growth) for prolonged periods. When necessary, however (such as during wound repair), these cells can undergo rapid proliferation and turnover within a short period of time. Although angiogenesis is a highly regulated process under normal conditions, many conditions (characterized as “angiogenic diseases”) are driven by persistent unregulated angiogenesis. Otherwise stated, unregulated angiogenesis may either cause a particular pathological condition directly or exacerbate an existing pathological condition. For example, ocular neovascularization has been implicated as the most common cause of blindness and dominates approximately twenty eye diseases. In certain existing conditions, such as arthritis, newly formed capillary blood vessels invade the joints and destroy cartilage. In diabetes, new capillaries formed in the retina invade the vitreous, bleed, and cause blindness. Growth and metastasis of tumors are also angiogenesis-dependent (Folkman, J., Cancer Research, 46:467-473, 1986; Folkman, J., Journal of the National Cancer Institute, 82:4-6, 1989). It has been shown, for example, that tumors which enlarge to greater than 2 mm, must obtain their own blood supply and do so by inducing the growth of new capillary blood vessels. Once these new blood vessels become embedded in the tumor, they provide a means for tumor cells to enter the circulation and metastasize to distant site, such as liver, lung or bone (Weidner, N. et al., The New England Journal of Medicine, 324(1):1-8, 1991).

The pharmaceutical compositions of the subject invention can be formulated according to known methods for preparing pharmaceutically useful compositions. Formulations are described in a number of sources which are well known and readily available to those skilled in the art. For example, Remington's Pharmaceutical Sciences (Martin E W 1995 Easton Pa. Mack Publishing Company, 19.sup.th ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations maybe presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In one embodiment, the Aβ peptide fragments are delivered in a sustained release formulation. The formulations provide extended release and extended half-life. Controlled release systems suitable for use include, without limitation, diffusion-controlled, solvent-controlled and chemically-controlled systems. Diffusion controlled systems include, for example reservoir devices, in which the Aβ peptide fragment or fragments are enclosed within a device such that release of the peptide fragments is controlled by permeation through a difussion barrier. Common reservoir devices include, for example, membranes, capsules, microcapsules, liposomes, and hollow fibers. Monolithic (matrix) device are a second type of diffusion controlled system, wherein the Aβ peptide fragment(s) are dispersed or dissolved in an rate-controlling matrix (e.g., a polymer matrix). The peptide fragments are homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

The peptide fragments of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

In certain embodiments of this invention, the complex comprises a liposome with a peptide fragment associated with the surface of the liposome or encapsulated within the liposome. Preformed liposomes can be modified to associate with the peptide fragments. For example, a cationic liposome associates through electrostatic interactions with the peptide fragment. Alternatively, a peptide fragment attached to a lipophilic compound, such as cholesterol, can be added to preformed liposomes whereby the cholesterol becomes associated with the liposomal membrane. Alternatively, the peptide fragment can be associated with the liposome during the formulation of the liposome.

Sterically stabilized liposomes of small diameter have the insoluble peptide fragment incorporated into the hydrophobic region of the lipid bilayer, which serves to significantly increase solubility of the peptide fragment and protect it from degradation or removal from circulation. Micelle technology utilizes sterically stabilized micelles where the insoluble peptide is actually coated with individual lipids through hydrophobic interaction with the hydrocarbon tail of the lipids, leaving the polar head of the lipid to interact with the aqueous environment. Liposomes can encapsulate micelles containing an insoluble peptide fragment with the advantage of higher stability and doses reaching the target. These liposomes, micelles, and micelle-containing liposomes can be modified to include a water-soluble polymer, such as polyethylene glycol (PEG), which reduces the rate by which the micelle and liposome are removed from circulation by the RES uptake and also increases the water solubility of the micelle or liposome, prolonging circulatory half life and bioactivity.

Solid lipid nanoparticles (SLNs) can also be used as alternative drug delivery systems to colloidal delivery systems such as lipid emulsions, liposomes, and polymeric nanoparticles. Various lipid matrices, surfactants, and other excipients used in formulation, preparation methods, sterilization and lyophilization of SLNs can be used. Entrapment efficiency of carrier and its effect on physical parameters, peptide release, and release mechanisms of various compositions are reviewed and discussed in Manjunath, et al. (2005) Methods Find Exp Clin Pharmacol 27(2): 127.

Therapeutically effective and optimal dosage ranges for the Aβ peptide fragments can be determined using methods known in the art. Guidance as to appropriate dosages to achieve an anti-angiogenesis and/or anti-tumor effect is provided from the exemplified assays disclosed herein. The minimal amounts of Aβ peptide fragment to achieve a therapeutic effect can likewise be determined. In one embodiment, the Aβ peptide fragment is administered in an equivalent amount to be within the μM dose range. In another embodiment, an amount equivalent to about 1 μM to about 100 μM Aβ peptide fragment is administered. In another embodiment, an amount equivalent to about 2 μM to about 10 μM Aβ peptide fragment is administered. Pharmaceutical formulations that can be administered can comprise, e.g., 1-10,000 mg, 10-1000 mg, 50-900 mg, 100-800 mg, or 200-500 mg.

The subject invention also pertains to diagnostic and/or screening methods and kits to screen for compounds that are potentially therapeutic in treatment of Alzheimer's disease by interfering with the anti-angiogenic effect of an Aβ peptide fragment.

In one aspect, included is a method for identifying compounds that interfere with Aβ-induced angiogenesis inhibition, wherein the method includes the steps of (a) contacting a first biological sample capable of undergoing angiogenesis with a test compound, a biologically active amount of an Aβ peptide fragment, and an angiogenic agent; and (b) determining the extent of angiogenesis that occurs in the first biological sample. Optionally, the method can include the steps of: (c) separately contacting a second biological sample capable of undergoing angiogenesis with a biologically active amount of an Aβ peptide fragment and an angiogenic agent; (d) determining the extent of angiogenesis that occurs in the second biological sample; and (e) comparing the extent of angiogenesis that occurs in the first biological sample with that which occurs in the second biological sample. In this way, steps (c)-(d) can be utilized as a control. Preferably, the same Aβ peptide fragment is used in the first and second biological samples.

Determining the extent of angiogenesis can be carried out using methods known in the art, such as those described herein, and can be done qualitatively or quantitatively. For example, molecular or cellular markers of cancer or tumor growth can be utilized. The extent of angiogenesis can also be determined by measuring the amount of endothelial cell proliferation or the extent of blood vessel growth within a biological sample.

The biological samples utilized in the methods and kits can include various biological fluids and tissues that can exhibit angiogenesis and/or tumor development. For example, the biological sample can be arterial tissue, corneal tissue, endothelial cells, umbilical cord tissue, chorionic allantoid membrane, and the like.

The angiogenic agent can be any molecule, compound, or cell that is capable of inducing angiogenesis in the biological sample. For example, the angiogenic agent can be a trophic factor, such as a neurotrophic factor. The angiogenic factor can be a cytokine or growth factor such as vascular endothelial growth factor, platelet-derived growth factor, and basic fibroblast growth factor. The diagnostic and/or screening methods of the subject invention can be carried out in vivo, such as in an animal model, or in vitro.

In another aspect, the subject invention includes a kit for identifying compounds that interfere with Aβ-induced angiogenesis inhibition. The kit can include a compartment containing at least one Aβ peptide fragment and, optionally, a compartment containing an angiogenic agent. Furthermore, the kit can optionally include a compartment containing one or more biological samples.

In another aspect, a method is provided for identifying compounds that interfere with Aβ-induced anti-tumor activity, including the steps of: (a) contacting a first tumor tissue with a test compound and a biologically active amount of an Aβ peptide fragment; and (b) determining the extent of tumor growth that occurs in the tumor tissue. Optionally, the method can further include the steps of: (c) separately contacting a second tumor tissue with a biologically active amount of an Aβ peptide fragment; (d) determining the extent of tumor growth that occurs in the second tumor tissue; and (e) comparing the extent of tumor growth that occurs in the first tumor tissue with that which occurs in the second tumor tissue. In this way, steps (c)-(d) can be utilized as a control. The extent of tumor growth can be determined quantitatively or qualitatively using methods known in the art, including methods described herein. For example, molecular or cellular markers of cancer or tumor growth can be utilized.

In another aspect, the subject invention includes a kit for identifying compounds that interfere with Aβ-induced anti-tumor activity. The kit can include a compartment containing at least one Aβ peptide fragment and, optionally, a compartment containing at least one tumor tissue. Furthermore, the kit can optionally include a compartment containing one or more biological samples.

The test compounds that can be screened using the methods and kits of the subject invention can include any substance, agent, or molecule, including, for example, small molecules and living or dead cells.

A variety of patients may be treated including any vertebrate species. Preferably, the patient is of a mammalian species. Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales.

### Treatment of Tumors and Cancer

In one embodiment, a method for treating tumors, cancers or other proliferative disorders in animals or humans in need of such treatment is provided, comprising administering a therapeutically effective amount, optionally in unit dosage form, of an Aβ peptide fragment described herein. Also provided are methods for inhibiting angiogenesis in animals or humans in need thereof, comprising administering a therapeutically effective amount, optionally in unit dosage form, of an Aβ peptide fragment disclosed herein.

Aβ peptide fragments and pharmaceutical compositions comprising the fragments, are provided, that can be used in one embodiment to treat tumors and cancers, including, but not limited to cancers or tumors in the following tissues or organs: breast, prostate, lung, bronchus, colon, urinary tract, bladder, kidney, pancreas, thyroid, stomach, brain, esophagus, liver, intrahepatic bile duct, cervix, skin, larynx, heart, testis, small intestine, thyroid, vulva, gallbladder, pleura, eye, nose, ear, nasopharnx, ureter, gastrointestineal system, rectal tissue, pancreas, head and neck. Cancers that can be treated include without limitation non-Hodgkin lymphoma, melanoma, multiple myeloma, acute myeloid leukemia, chronic lymphatic leukemia, Hodgkin lymphoma, chronic myeloid leukemia, acute lymphatic leukemia, carcinomas, adenocarcinomas; sarcomas; lymphomas, and leukemias.

In one subembodiment, the Aβ peptide fragments can be used to treat, for example, prostate cancer, lung cancer, colorectal cancer, bladder cancer, cutaneous melanoma, pancreatic cancer, leukemia, breast cancer, endometrial cancer, non-Hodgkin's lymphoma, and ovarian cancer.

In another subembodiment, the Aβ peptide fragments can be used to treat epithelial cell cancers and tumors including: skin cancer, cervical cancer, anal carcinoma, esophageal cancer, hepatocellular carcinoma (in the liver), laryngeal cancer, renal cell carcinoma (in the kidneys), stomach cancer, testicular cancers, and thyroid cancer.

In another subembodiment, the Aβ peptide fragments are used to treat hematological malignancies (blood and bone marrow) including leukemia, lymphoma, and multiple myeloma.

In a further subembodiment, the Aβ peptide fragments are used to treat sarcomas including: osteosarcoma (in bone), chondrosarcoma (arising from cartilage), and rhabdomyosarcoma (in muscle).

In another subembodiment, the Aβ peptide fragments are used to treat cancers and tumors of miscellaneous origin including: brain tumors, gastrointestinal stromal tumors (GIST), mesothelioma (in the pleura or pericardium), thymoma and teratomas, and melanoma.

Examples of tumors that can be treated include, without limitation, malignant brain tumors, such as glioblastomas; malignant lung tumors, such as adenocarcinomas; or malignant tumors of the breast, colon, kidney, bladder, head or neck.

Proliferative disorders that can be treated include, without limitation, hematopoietic disorders, such as leukemias, lymphomas or polycythemias; and ocular disorders, such as diabetic retinopathy, macular degeneration, glaucoma or retinitis pigmentosa. Inflammatory disorders that can be treated include, without limitation, rheumatoid arthritis, osteoarthritis, pulmonary fibrosis, sarcoid granulomas, psoriasis or asthma.

In one embodiment, the Aβ peptide fragments can be used to treat a carcinoma, sarcoma, lymphoma, leukemia, and/or myeloma. In other embodiments, the Aβ peptide fragments disclosed herein can be used to treat solid tumors.

In other embodiments, the Aβ peptide fragments described herein can be used for the treatment of cancer, including, but not limited to the cancers listed in Table 2a below.

TABLE 2a
Types of Cancer

Acute Lymphoblastic Leukemia, Adult
Acute Lymphoblastic Leukemia, Childhood
Acute Myeloid Leukemia, Adult
Acute Myeloid Leukemia, Childhood
Adrenocortical Carcinoma
Adrenocortical Carcinoma, Childhood
AIDS-Related Cancers
AIDS-Related Lymphoma
Anal Cancer
Astrocytoma, Childhood Cerebellar
Astrocytoma, Childhood Cerebral
Basal Cell Carcinoma
Bile Duct Cancer, Extrahepatic
Bladder Cancer
Bladder Cancer, Childhood
Bone Cancer, Osteosarcoma/Malignant Fibrous Histiocytoma
Brain Stem Glioma, Childhood
Brain Tumor, Adult
Brain Tumor, Brain Stem Glioma, Childhood
Brain Tumor, Cerebellar Astrocytoma, Childhood
Brain Tumor, Cerebral Astrocytoma/Malignant Glioma, Childhood
Brain Tumor, Ependymoma, Childhood
Brain Tumor, Medulloblastoma, Childhood
Brain Tumor, Supratentorial Primitive Neuroectodermal Tumors,
Childhood
Brain Tumor, Visual Pathway and Hypothalamic Glioma, Childhood
Brain Tumor, Childhood
Breast Cancer
Breast Cancer, Childhood
Breast Cancer, Male
Bronchial Adenomas/Carcinoids, Childhood
Burkitt's Lymphoma
Carcinoid Tumor, Childhood
Carcinoid Tumor, Gastrointestinal
Carcinoma of Unknown Primary
Central Nervous System Lymphoma, Primary
Cerebellar Astrocytoma, Childhood
Cerebral Astrocytoma/Malignant Glioma, Childhood
Cervical Cancer
Childhood Cancers
Chronic Lymphocytic Leukemia
Chronic Myelogenous Leukemia
Chronic Myeloproliferative Disorders
Colon Cancer
Colorectal Cancer, Childhood
Cutaneous T-Cell Lymphoma, see Mycosis Fungoides and Sézary
Syndrome
Endometrial Cancer
Ependymoma, Childhood
Esophageal Cancer
Esophageal Cancer, Childhood
Ewing's Family of Tumors
Extracranial Germ Cell Tumor, Childhood
Extragonadal Germ Cell Tumor
Extrahepatic Bile Duct Cancer
Eye Cancer, Intraocular Melanoma
Eye Cancer, Retinoblastoma
Gallbladder Cancer
Gastric (Stomach) Cancer
Gastric (Stomach) Cancer, Childhood
Gastrointestinal Carcinoid Tumor
Germ Cell Tumor, Extracranial, Childhood
Germ Cell Tumor, Extragonadal
Germ Cell Tumor, Ovarian
Gestational Trophoblastic Tumor
Glioma, Adult
Glioma, Childhood Brain Stem
Glioma, Childhood Cerebral Astrocytoma
Glioma, Childhood Visual Pathway and Hypothalamic
Skin Cancer (Melanoma)
Skin Carcinoma, Merkel Cell
Small Cell Lung Cancer
Small Intestine Cancer
Soft Tissue Sarcoma, Adult
Soft Tissue Sarcoma, Childhood
Squamous Cell Carcinoma, see Skin Cancer (non-Melanoma)
Squamous Neck Cancer with Occult Primary, Metastatic
Stomach (Gastric) Cancer
Stomach (Gastric) Cancer, Childhood
Supratentorial Primitive Neuroectodermal Tumors, Childhood
T-Cell Lymphoma, Cutaneous, see Mycosis Fungoides and Sézary
Syndrome
Testicular Cancer
Thymoma, Childhood
Thymoma and Thymic Carcinoma
Thyroid Cancer
Thyroid Cancer, Childhood
Transitional Cell Cancer of the Renal Pelvis and Ureter
Trophoblastic Tumor, Gestational
Unknown Primary Site, Carcinoma of, Adult
Unknown Primary Site, Cancer of, Childhood
Unusual Cancers of Childhood
Ureter and Renal Pelvis, Transitional Cell Cancer
Urethral Cancer
Uterine Cancer, Endometrial
Uterine Sarcoma
Vaginal Cancer
Visual Pathway and Hypothalamic Glioma, Childhood
Vulvar Cancer
Waldenström's Macroglobulinemia
Wilms' Tumor
Hairy Cell Leukemia
Head and Neck Cancer
Hepatocellular (Liver) Cancer, Adult (Primary)
Hepatocellular (Liver) Cancer, Childhood (Primary)
Hodgkin's Lymphoma, Adult
Hodgkin's Lymphoma, Childhood
Hodgkin's Lymphoma During Pregnancy
Hypopharyngeal Cancer
Hypothalamic and Visual Pathway Glioma, Childhood
Intraocular Melanoma
Islet Cell Carcinoma (Endocrine Pancreas)
Kaposi's Sarcoma
Kidney (Renal Cell) Cancer
Kidney Cancer, Childhood
Laryngeal Cancer
Laryngeal Cancer, Childhood
Leukemia, Acute Lymphoblastic, Adult
Leukemia, Acute Lymphoblastic, Childhood
Leukemia, Acute Myeloid, Adult
Leukemia, Acute Myeloid, Childhood
Leukemia, Chronic Lymphocytic
Leukemia, Chronic Myelogenous
Leukemia, Hairy Cell
Lip and Oral Cavity Cancer
Liver Cancer, Adult (Primary)
Liver Cancer, Childhood (Primary)
Lung Cancer, Non-Small Cell
Lung Cancer, Small Cell
Lymphoma, AIDS-Related
Lymphoma, Burkitt's
Lymphoma, Cutaneous T-Cell, see Mycosis Fungoides and Sézary
Syndrome
Lymphoma, Hodgkin's, Adult
Lymphoma, Hodgkin's, Childhood
Lymphoma, Hodgkin's During Pregnancy
Lymphoma, Non-Hodgkin's, Adult
Lymphoma, Non-Hodgkin's, Childhood
Lymphoma, Non-Hodgkin's During Pregnancy
Lymphoma, Primary Central Nervous System
Macroglobulinemia, Waldenström's
Malignant Fibrous Histiocytoma of Bone/Osteosarcoma
Medulloblastoma, Childhood
Melanoma
Melanoma, Intraocular (Eye)
Merkel Cell Carcinoma
Mesothelioma, Adult Malignant
Mesothelioma, Childhood
Metastatic Squamous Neck Cancer with Occult Primary
Multiple Endocrine Neoplasia Syndrome, Childhood
Multiple Myeloma/Plasma Cell Neoplasm
Mycosis Fungoides
Myelodysplastic Syndromes
Myelodysplastic/Myeloproliferative Diseases
Myelogenous Leukemia, Chronic
Myeloid Leukemia, Adult Acute
Myeloid Leukemia, Childhood Acute
Myeloma, Multiple
Myeloproliferative Disorders, Chronic
Nasal Cavity and Paranasal Sinus Cancer
Nasopharyngeal Cancer
Nasopharyngeal Cancer, Childhood
Neuroblastoma
Non-Hodgkin's Lymphoma, Adult
Non-Hodgkin's Lymphoma, Childhood
Non-Hodgkin's Lymphoma During Pregnancy
Non-Small Cell Lung Cancer
Oral Cancer, Childhood
Oral Cavity Cancer, Lip and Oropharyngeal Cancer
Osteosarcoma/Malignant Fibrous Histiocytoma of Bone
Ovarian Cancer, Childhood
Ovarian Epithelial Cancer
Ovarian Germ Cell Tumor
Ovarian Low Malignant Potential Tumor
Pancreatic Cancer
Pancreatic Cancer, Childhood
Pancreatic Cancer, Islet Cell
Paranasal Sinus and Nasal Cavity Cancer
Parathyroid Cancer
Penile Cancer
Pheochromocytoma
Pineoblastoma and Supratentorial Primitive Neuroectodermal Tumors,
Childhood
Pituitary Tumor
Plasma Cell Neoplasm/Multiple Myeloma
Pleuropulmonary Blastoma
Pregnancy and Breast Cancer
Pregnancy and Hodgkin's Lymphoma
Pregnancy and Non-Hodgkin's Lymphoma
Primary Central Nervous System Lymphoma
Prostate Cancer
Rectal Cancer
Renal Cell (Kidney) Cancer
Renal Cell (Kidney) Cancer, Childhood
Renal Pelvis and Ureter, Transitional Cell Cancer
Retinoblastoma
Rhabdomyosarcoma, Childhood
Salivary Gland Cancer
Salivary Gland Cancer, Childhood
Sarcoma, Ewing's Family of Tumors
Sarcoma, Kaposi's
Sarcoma, Soft Tissue, Adult
Sarcoma, Soft Tissue, Childhood
Sarcoma, Uterine
Sezary Syndrome
Skin Cancer (non-Melanoma)
Skin Cancer, Childhood

### Anti-Angiogenic Activity of the Aβ Peptide Fragment

Without being limited to any theory, it is possible that the sequence HHQKLVFF is the sequence of Aβ that confers anti-antiogenic activity.

Numerous studies have shown that heparin and various proteoglycans on the cell surface can bind to Aβ peptides (Snow, et al. 1995 Arch. Biochem. Biophys. 320, 84-95; McLaurin, et al. 2000 Eur. J. Biochem. 267, 6353-61; McKeon J, Holland L A. 2004 Electrophoresis 25, 1243-8), and heparan sulfate proteoglycans have been shown to be associated with amyloid deposits in AD brain (van Horssen, et al. 2001 Acta Neuropathol. (Berl). 102, 604-14). Heparin sulfate proteoglycans also play a prominent role during angiogenesis by allowing the interaction of specific growth factors such as basic fibroblast growth factor (bFGF) and vascular endothelial growth factor (VEGF) with the cell surface. In this way, proteoglycans are thought to modulate the interaction of growth factors with receptors (Rusnati M, Presta M. 1996 Int. J. Clin. Lab. Res. 26, 15-23; Dougher, et al. 1997 Growth Factors. 14, 257-68). It is shown herein that the addition of exogenous heparin is able to effectively reverse the anti-angiogenic activity of Aβ₁₋₄₂. Addition of heparin alone caused a slight inhibition of angiogenesis, which is consistent with studies indicating the inhibitory effect of excess heparins on angiogenesis. The mechanism of this effect has been suggested to be via an increased release of tissue factor pathway inhibitor (Mousa S A, Mohamed S. 2004 Thromb. Haemost. 92, 627-33).

Cell surface proteoglycans such as heparan sulfate proteoglycans can bind to and allow the activity of various growth factors including VEGF and bFGF (Iozzo R V, San Antonio J D. 2001 J. Clin. Invest. 108, 349-55; Presta, et al. 2005 Cytokine Growth Factor Rev. 16, 159-78; Sanderson, et al. 2005 J. Cell Biochem. September 7, (advance electronic publication)). It is possible that Aβ binds to these proteoglycans, impacting the binding and interaction of growth factors with the cell. Therefore, as the angiogenesis assays contain heparin binding growth factors, the addition of excess heparin may act to bind out Aβ peptides and prevent their binding to the cell surface, hence opposing the anti-angiogenic activity of Aβ. Alternatively, Aβ has also been shown to directly interact with the heparin binding motif on VEGF (Yang, et al. 2005 J. Neurochem. 93, 118-27); hence it is possible that the binding of Aβ to heparin can prevent it from binding to VEGF, reversing the anti-angiogenic activity of Aβ. It is also possible that heparin (and other glycosaminoglycans) affect the conformational properties of Aβ peptides, changing the rate of fibril formation (Castillo, et al. 1999 J. Neurochem. 72, 1681-7; Cohlberg, et al. 2002 Biochemistry. 41, 1502-11) thereby rendering the peptide unable to block angiogenesis. The anti-angiogenic activity of Aβ peptides in-vitro seems to be related to their conformational properties, as preparations of Aβ containing higher n-sheet content are more potently anti-angiogenic (Gebbink, et al. 2000 Biochim. Biophys. Acta. 1502, 16-30. Additionally, soluble oligomers of the peptide are particularly anti-angiogenic whereas fibrillar forms are inactive (Paris, et al. 2005 Brain Res. Mol. Brain Res. 136, 212-30; Skovseth, et al. 2005 Blood 105, 1044-51) suggesting that particular residues in the Aβ peptide need to be exposed in order to inhibit angiogenesis.

One motif within the Aβ peptide sequence which may be important for imparting anti-angiogenic activity is the putative proteoglycan binding region, HHQK (Cardin, A. D.; Weintaub, H. J. R. 1989 Arteriosclerosis. 9, 21-32; Snow, et al. 1995 Arch. Biochem. Biophys. 320, 84-95; McLaurin, et al. 2000 Eur. J. Biochem. 267, 6353-61; McKeon, et al. 2004 Electrophoresis. 25, 1243-8). Proteoglycans are known to play a regulatory role during angiogenesis (Moon, et al. 2005 J. Cell Physiol. 203, 166-76; Tkachenko, et al. 2005 Circ. Res. 96, 488-500; Presta, et al. 2005) Cytokine Growth Factor Rev. 16, 159-78). Also, numerous studies have indicated an important role for heparan sulfate proteoglycans in AD pathogenesis, and it has been suggested that interference with the binding of these molecules to Aβ may be beneficial therapeutically (Leveugle, et al. 1994) Neuroreport. 5, 1389-92; Kisilevsky, et al. 2002 J. Mol. Neurosci. 19, 45-50).

Another potentially significant sequence for anti-angiogenic activity is the four amino acids adjacent to the HHQK motif, towards the C-terminal portion (LVFF). This region is known to constitute part of the β strand and hence is important for oligomerization of the peptide (Morimoto, et al. 2004 J. Biol. Chem. 279, 52781-8; hie, et al. 2005 J. Biosci. Bioeng. 99, 437-47). It has recently been shown in a conformational model of Aβ₁₀₋₄₂ that the highly hydrophobic residues 17-20 are exposed in the dimeric form, while studies by another group reveal that this region is buried in the fibrillar form (Mathura, et al. 2005 Biochem. Biophys. Res. Commun. 332, 585-92; Olofsson, et al. 2005 J. Biol. Chem. October 7, (advance electronic publication)).

To further investigate the possibility that Aβ could be acting by preventing the binding of growth factors to proteoglycans on the cell surface, three residues were substituted in the putative proteoglycan binding sequence, HHQK of Aβ₁₂₋₂₈. It is shown herein that the neutral amino acid substitutions GGQG or AAQA in place of the wildtype HHQK completely abolish the anti-angiogenic affect of the wildtype Aβ₁₂₋₂₈ peptide. Further, the anti-angiogenic potency of Aβ₁₂₋₂₈ in-vivo was confirmed by using a rat corneal micro-pocket model of VEGF-induced angiogenesis. Levels of VEGF are increased in the brain of AD patients (Kalaria, et al. 1998 Brain Res. Mol. Brain Res. 62, 101-5; Tarkowski, et al. 2002 Neurobiol Aging. 23, 237-43), but this is not associated with an increased brain vascularization (Buee, et al. 1997 Ann. N.Y. Acad. Sci. 826, 7-24). The accumulation of Aβ in AD brains may therefore result in the inhibition of VEGF activity. VEGF is neurotrophic, it is important for maintaining vascular integrity, and also a key factor in vascular remodeling following stroke or head injury (Slevin, et al. 2000 Neuroreport 11, 2759-64; Shore, et al. 2004 Neurosurgery. 54, 605-12). The antagonistic action of Aβ towards VEGF in the AD brain may explain why AD patients and transgenic mouse models of AD do poorly following stroke (Koistinaho, et al. 2002 Proc. Natl. Acad. Sci. U.S.A. 99, 1610-5; Wen, et al. 2004 J. Biol. Chem. 279, 22684-92; Koistinaho M, Koistinaho J. 2005 Brain Res. Brain Res. Rev. 48, 240-50).

Examples provided herein support that the proteoglycan binding motif alone may not be sufficient to elicit anti-angiogenic effects, and that the amino acids immediately adjacent to this sequence (LVFF) are required to mediate the anti-angiogenic activity of Aβ. In a conformational model of Aβ oligomers, it has been shown that the LVFF sequence (amino acids 17-20) is an exposed region of the peptide (Mathura, et al. 2005 Biochem. Biophys. Res. Commun. 332, 585-92).

The pro-angiogenic affects of Aβ₃₄₋₄₂ have also been noted. The pro-angiogenic activity of the Aβ₃₄₋₄₂ fragment observed in the network assay described herein is consistent with the pro-angiogenic activity of Aβ_{1-40/42} peptides at low concentrations that has previously observed (Paris, et al. 2004 Angiogenesis. 7, 75-85; Cantara, et al. 2004 F.A.S.E.B. J. 18, 1943-5). The folding of Aβ may be such that the C-terminal 34-42 sequence is left exposed when monomers and dimers are formed. Subsequently this region may be buried upon higher order oligomer or fibril formation. A recent NMR study of Aβ₁₋₄₂ fibrils confirmed that the residues 28-42 are solvent inaccessible and the back bone amides were not amenable for a deuterium exchange even after a long time period (Olofsson, et al. 2005 J. Biol. Chem. October 7, (advance electronic publication)). Thus, the pro-angiogenic effect of Aβ_{1-40/42} peptides at low concentrations may be due to their predominantly monomeric or dimeric states exposing a pro-angiogenic motif (Olofsson, et al. 2005 J. Biol. Chem. October 7, (advance electronic publication); Fraser, et al. 1994 J. Mol. Biol. 244, 64-73; van Horssen, et al. 2001 Acta Neuropathol. (Berl). 102, 604-14; Rusnati, M; Presta, M. 1996 Int. J. Clin. Lab. Res. 26, 15-23; Dougher, et al. 1997 Growth Factors. 14, 257-68; Mousa, et al. 2004 Thromb. Haemost. 92, 627-33; Iozzo, et al. 2001 J. Clin. Invest. 108, 349-55; Presta, et al. 2005 Cytokine Growth Factor Rev. 16, 159-78; Sanderson, et al. 2005 J. Cell Biochem. September 7, (advance electronic publication)) in the C-terminal region.

### Combination Therapy

In one aspect, the peptide fragments disclosed herein can be used in combination with at least one additional chemotherapeutic agent in order to treat a cancer, tumor or other proliferative disorder. The additional agents can be administered in combination or alternation with the compounds disclosed herein. The drugs can form part of the same composition, or be provided as a separate composition for administration at the same time or a different time.

Examples of second therapeutic agents include but are not limited to, IL-12, retinoids, interferons, angiostatin, endostatin, thalidomide, thrombospondin-1, thrombospondin-2, captopryl, anti-neoplastic agents such as alpha interferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG and radiation.

Other example include agents with antimitotic effects (antimitotic inhibitors), such as those which target cytoskeletal elements, including microtubule modulators such as taxane drugs (such as taxol, paclitaxel, taxotere, docetaxel), podophylotoxins or vinca alkaloids (vincristine, vinblastine); antimetabolite drugs (such as 5-fluorouracil, cytarabine, gemcitabine, purine analogues such as pentostatin, methotrexate); alkylating agents or nitrogen mustards (such as nitrosoureas, cyclophosphamide or ifosphamide); drugs which target DNA such as the antracycline drugs adriamycin, doxorubicin, pharmorubicin or epirubicin; drugs which target topoisomerases (topoisomerase inhibitors) such as etoposide; hormones and hormone agonists or antagonists such as estrogens, antiestrogens (tamoxifen and related compounds) and androgens, flutamide, leuprorelin, goserelin, cyprotrone or octreotide; drugs which target signal transduction in tumor cells including antibody derivatives such as herceptin; alkylating drugs such as platinum drugs (cis-platin, carbonplatin, oxaliplatin, paraplatin) or nitrosoureas; drugs potentially affecting metastasis of tumours such as matrix metalloproteinase inhibitors; gene therapy and antisense agents; antibody therapeutics; other bioactive compounds of marine origin, such as the didemnins such as aplidine; corticosteroids; steroid analogues, such as dexamethasone; anti-inflammatory drugs, including nonsteroidal agents (such as acetaminophen or ibuprofen) or steroids and their derivatives in particular dexamethasone; and anti-emetic drugs, including 5HT-3 inhibitors (such as gramisetron or ondasetron).

Other examples of second therapeutic agents include those disclosed below in Table 1a.

TABLE 1a
Chemotherapeutic Agents

13-cis-Retinoic Acid
2-Amino-6-Mercaptopurine
2-CdA
2-Chlorodeoxyadenosine
5-fluorouracil
5-FU
6-TG
6-Thioguanine
6-Mercaptopurine
6-MP
Accutane
Actinomycin-D
Adriamycin
Adrucil
Agrylin
Ala-Cort
Aldesleukin
Alemtuzumab
Alitretinoin
Alkaban-AQ
Alkeran
All-transretinoic acid
Alpha interferon
Altretamine
Amethopterin
Amifostine
Aminoglutethimide
Anagrelide
Anandron
Anastrozole
Arabinosylcytosine
Ara-C
Aranesp
Aredia
Arimidex
Aromasin
Arsenic trioxide
Asparaginase
ATRA
Avastin
BCG
BCNU
Bevacizumab
Bexarotene
Bicalutamide
BiCNU
Blenoxane
Bleomycin
Bortezomib
Busulfan
Busulfex
C225
Calcium Leucovorin
Campath
Camptosar
Camptothecin-11
Capecitabine
Carac
Carboplatin
Carmustine
Carmustine wafer
Casodex
CCNU
CDDP
CeeNU
Cerabidine
cetuximab
Chlorambucil
Cisplatin
Citrovorum Factor
Cladribine
Cortisone
Cosmegen
CPT-11
Cyclophosphamide
Cytadren
Cytarabine
Cytarabine liposomal
Cytosar-U
Cytoxan
Dacarbazine
Dactinomycin
Darbepoetin alfa
Daunomycin
Daunorubicin
Daunorubicin hydrochloride
Daunorubicin liposomal
DaunoXome
Decadron
Delta-Cortef
Deltasone
Denileukin diftitox
DepoCyt
Dexamethasone
Dexamethasone acetate
dexamethasone sodium phosphate
Dexasone
Dexrazoxane
DHAD
DIC
Diodex
Docetaxel
Doxil
Doxorubicin
Doxorubicin liposomal
Droxia
DTIC
DTIC-Dome
Duralone
Efudex
Eligard
Ellence
Eloxatin
Elspar
Emcyt
Epirubicin
Epoetin alfa
Erbitux
Erwinia L-asparaginase
Estramustine
Ethyol
Etopophos
Etoposide
Etoposide phosphate
Eulexin
Evista
Exemestane
Fareston
Faslodex
Femara
Filgrastim
Floxuridine
Fludara
Fludarabine
Fluoroplex
Fluorouracil
Fluorouracil (cream)
Fluoxymesterone
Flutamide
Folinic Acid
FUDR
Fulvestrant
G-CSF
Gefitinib
Gemcitabine
Gemtuzumab ozogamicin
Gemzar
Gleevec
Lupron
Lupron Depot
Matulane
Maxidex
Mechlorethamine
Mechlorethamine Hydrochlorine
Medralone
Medrol
Megace
Megestrol
Megestrol Acetate
Melphalan
Mercaptopurine
Mesna
Mesnex
Methotrexate
Methotrexate Sodium
Methylprednisolone
Mylocel
Letrozole
Neosar
Neulasta
Neumega
Neupogen
Nilandron
Nilutamide
Nitrogen Mustard
Novaldex
Novantrone
Octreotide
Octreotide acetate
Oncospar
Oncovin
Ontak
Onxal
Oprevelkin
Orapred
Orasone
Oxaliplatin
Paclitaxel
Pamidronate
Panretin
Paraplatin
Pediapred
PEG Interferon
Pegaspargase
Pegfilgrastim
PEG-INTRON
PEG-L-asparaginase
Phenylalanine Mustard
Platinol
Platinol-AQ
Prednisolone
Prednisone
Prelone
Procarbazine
PROCRIT
Proleukin
Prolifeprospan 20 with Carmustine implant
Purinethol
Raloxifene
Rheumatrex
Rituxan
Rituximab
Roveron-A (interferon alfa-2a)
Rubex
Rubidomycin hydrochloride
Sandostatin
Sandostatin LAR
Sargramostim
Solu-Cortef
Solu-Medrol
STI-571
Streptozocin
Tamoxifen
Targretin
Taxol
Taxotere
Temodar
Temozolomide
Teniposide
TESPA
Thalidomide
Thalomid
TheraCys
Thioguanine
Thioguanine Tabloid
Thiophosphoamide
Thioplex
Thiotepa
TICE
Toposar
Topotecan
Toremifene
Trastuzumab
Tretinoin
Trexall
Trisenox
TSPA
VCR
Velban
Velcade
VePesid
Vesanoid
Viadur
Vinblastine
Vinblastine Sulfate
Vincasar Pfs
Vincristine
Vinorelbine
Vinorelbine tartrate
VLB
VP-16
Vumon
Xeloda
Zanosar
Zevalin
Zinecard
Zoladex
Zoledronic acid
Zometa
Gliadel wafer
Glivec
GM-CSF
Goserelin
granulocyte - colony stimulating factor
Granulocyte macrophage colony stimulating factor
Halotestin
Herceptin
Hexadrol
Hexalen
Hexamethylmelamine
HMM
Hycamtin
Hydrea
Hydrocort Acetate
Hydrocortisone
Hydrocortisone sodium phosphate
Hydrocortisone sodium succinate
Hydrocortone phosphate
Hydroxyurea
Ibritumomab
Ibritumomab Tiuxetan
Idamycin
Idarubicin
Ifex
IFN-alpha
Ifosfamide
IL-2
IL-11
Imatinib mesylate
Imidazole Carboxamide
Interferon alfa
Interferon Alfa-2b (PEG conjugate)
Interleukin-2
Interleukin-11
Intron A (interferon alfa-2b)
Leucovorin
Leukeran
Leukine
Leuprolide
Leurocristine
Leustatin
Liposomal Ara-C
Liquid Pred
Lomustine
L-PAM
L-Sarcolysin
Meticorten
Mitomycin
Mitomycin-C
Mitoxantrone
M-Prednisol
MTC
MTX
Mustargen
Mustine
Mutamycin
Myleran
Iressa
Irinotecan
Isotretinoin
Kidrolase
Lanacort
L-asparaginase
LCR

### Assays Useful for the Peptides Disclosed Herein

Angiogenesis assays known in the art may be used. See, for example, U.S. Patent Application 2003/0077261A1 to Paris, et al. wherein rat aortic ring, bovine, mouse and human angiogenesis assays are described.

Quantification of ring microvessel outgrowths as described, for example, in U.S. Patent Application 2003/0077261A1 to Paris, et al. may be used wherein ring cultures are photographed using a digital video camera linked to an OLYMPUS BX60 microscope and the outgrowth area is selectively measured and detected with the Image Pro Plus software.

Endothelial Cell Migration Assays, described in U.S. Patent Application 2003/0077261A1 to Paris, et al. may be used, where migration of human brain adult endothelial cells is evaluated using a modified Boyden chamber assay (BD BioCoat MATRIGEL Invasion Chamber), as described (Soker et al. 1998; Nakamura et al. 1997).

Nude Mouse Tumor Xenograft models as described, for example, in U.S. Patent Application 2003/0077261A1 to Paris, et al. may be used wherein A-549 (human lung adenocarcinoma) and U87-MG (human glioblastoma) cells are implanted into 8-week-old female nude mice. Tumors grown in the animals are measuring before, after and during treatment with Aβ peptides. On the termination day of each in vivo antitumor study, tumors are extracted and microvessels are quantified.

The invention will be understood in further detail in view of the following nonlimiting examples.

### EXAMPLES

### Materials and Methods are Provided in Examples 1-7

### Example 1

### Cell Culture and Reagents

All in-vitro experiments were performed using primary Human Umbilical Vein Endothelial Cells (HUVEC) at passages 3-4, purchased from American Tissue Type Culture Collection (ATCC, VA). Cells were cultured in F12K Medium (ATCC, VA) supplemented with 10% fetal bovine serum (Invitrogen, CA), 0.1 mg/ml Heparin and 0.03 mg/ml endothelial cell growth supplement (Sigma-Aldrich, MO). At all times, cells were maintained in a sterile cell culture incubator at 37° C. and 5% CO₂.

### Example 2

### Preparation of Aβ Peptides

All peptides were prepared by and purchased from Biosource, CA upon request. 1 mg of lyophilized peptides were dissolved in 1 ml of 1,1,1,3,3,3-hexafluoro-2-propanol (HFIP) in order to minimize formation of β-sheet structures and promote α-helical secondary structure. Peptides were allowed to air dry in a chemical fume hood for one hour, followed by further drying in a speed-vac (Thermo-Savant, NY) for 30 minutes. The resulting clear film was re-suspended in 100% dimethylsulfoxide (DMSO) to a concentration of 1 mM. Peptides were subsequently aliquoted and stored at −80° C.

### Example 3

### Capillary Tube Formation Assay

HUVEC (7.5×10⁴ cells/ml) in 500 μl of medium were seeded in 24-well plates, on top of a layer of Matrigel basement membrane matrix (Invitrogen, CA) in F12K medium (ATCC, VA) containing 4% serum (Invitrogen, CA), 0.1 mg/ml Heparin and 0.03 mg/ml endothelial cell growth supplement (Sigma-Aldrich, MO). Cells were incubated with peptides (or control conditions) for 24 hours. Control wells received the same volume of vehicle (DMSO) used to dilute the peptides. Network formation experiments were performed in triplicate, and at least 2 randomly chosen fields were photographed for each well using a 4× objective. Capillary length was measured using Image Pro Plus software (Media Cybernetic, Inc., MD).

### Example 4

### Cell Proliferation Assay

HUVEC (5×10³ cells per well) were seeded in a 96 well plate. Cells were incubated with peptides (or control conditions) for 24 hours. A quick cell proliferation assay was performed as per the manufacturer's protocol (Biovision Inc., CA).

### Example 5

### Cell Adhesion Assay

HUVEC (1×10⁴ cells per well) were seeded in a 96 well plate pre-coated with basement membrane protein complex. Cells were incubated with peptides (or control conditions) for 2.5 hours. For measurement of cell adhesion, the Innocyte cell adhesion assay was used (Calbiochem, CA) and the protocol followed as per the manufacturer's recommendations.

### Example 6

### Rat Corneal Micropocket Assay

This assay was carried out as described previously (Paris D, Townsend K, Quadros A, Humphrey J, Sun J, Brem S, Wotoczek-Obadia M, DelleDonne A, Patel N, Obregon D F, Crescentini R, Abdullah L, Coppola D, Rojiani A M, Crawford F, Sebti S M, Mullan M. (2004) Angiogenesis. 7, 75-85), using hydron pellets containing either VEGF (200 ng) alone, or in combination with different amounts of peptide fragments. The vascular growth response was measured seven days post implantation. The lengths and widths of vessel outgrowths were measured and the angiogenic index (AI) calculated using the formula L×W=AI. Rats were perfused with colloidal carbon, eyes enucleated and fixed in 10% buffered formalin. Corneas were removed under an Olympus dissecting microscope and mounted on glass slides with Crystal Mount media.

### Example 7

### Statistical Analysis

Statistical analyses were performed using ANOVA with post-hoc comparisons using Scheffe's or Bonferroni's using SPSS for Windows release 10.1.

### Example 8

### Effect of Aβ Peptide Fragments on Capillary Tube Formation

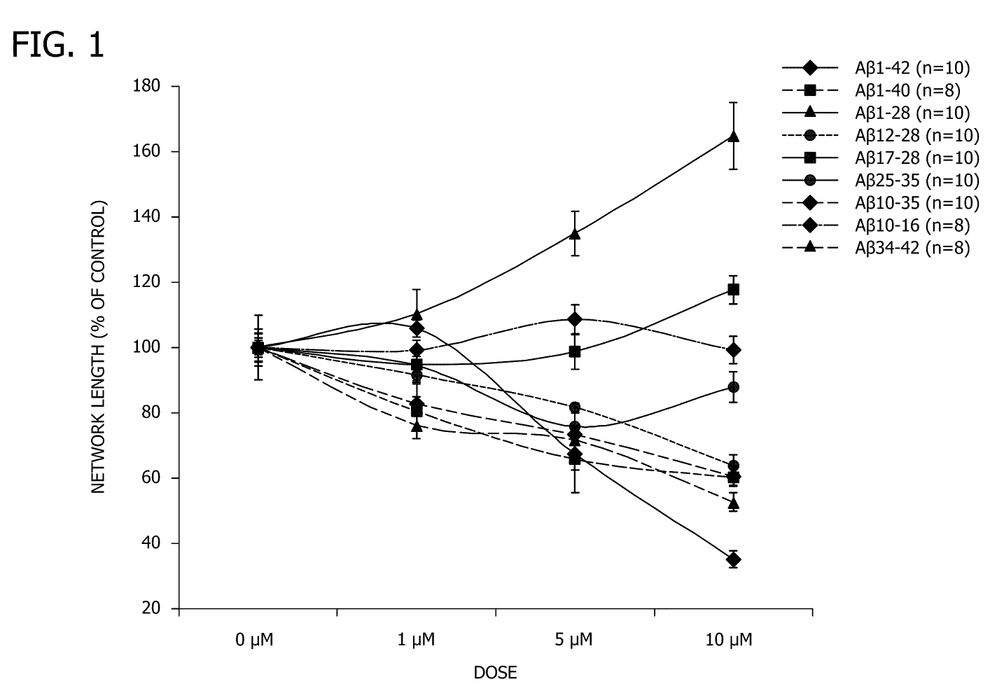

Various Aβ peptides and peptide fragments were tested for their ability to inhibit capillary network formation in the assay described in Example 3, including Aβ₁₋₄₂, Aβ₁₋₄₀, Aβ₁₋₂₈, Aβ₁₂₋₂₈, Aβ₁₇₋₂₈, Aβ₂₅₋₃₅, Aβ₁₀₋₃₅. Aβ₁₀₋₁₆ and Aβ₃₄₋₄₂) at 1, 5 and 10 μM. Total length of capillary tubes was quantified for each treatment group (n≧8), and expressed as a percentage of control treatment (FIG. 1).

Post hoc analysis revealed significant differences between control and Aβ₁₋₄₀, Aβ₁₋₄₂, Aβ₁₋₂₈, Aβ₁₀₋₃₅ and Aβ₁₂₋₂₈ at the 5 and 10 μM doses (P<0.005). Of the peptides tested, Aβ₁₋₂₈, Aβ₁₂₋₂₈, Aβ₁₀₋₃₅, Aβ₁₋₄₀ and Aβ₁₋₄₂ were the most active. Aβ₂₅₋₃₅ was slightly active at 5 μM, but not at 10 μM, and the other peptides (Aβ₁₀₋₁₆, Aβ₁₇₋₂₈ and Aβ₃₄₋₄₂) did not display any anti-angiogenic activity (FIG. 1). On the contrary, Aβ₃₄₋₄₂ promoted angiogenesis in a dose dependent manner. These data suggest that the minimal sequence required to preserve the anti-angiogenic activity of the Aβ peptide is included in residues 12-28. Furthermore, the observation that the Aβ₁₂₋₂₈ fragment is anti-angiogenic whereas the 17-28 fragment (missing the HHQK motif) is inactive suggests that the proteoglycan binding region (HHQK) present between residues 13-16 is required for anti-angiogenic activity.

### Example 9

### Effect of Aβ Peptide Fragments on Cell Proliferation and Cell Adhesion

Various Aβ peptide fragments were tested for their ability to inhibit cellular proliferation and cellular adhesion to a basement membrane complex using the assays described in Example 4 and 5, respectively.

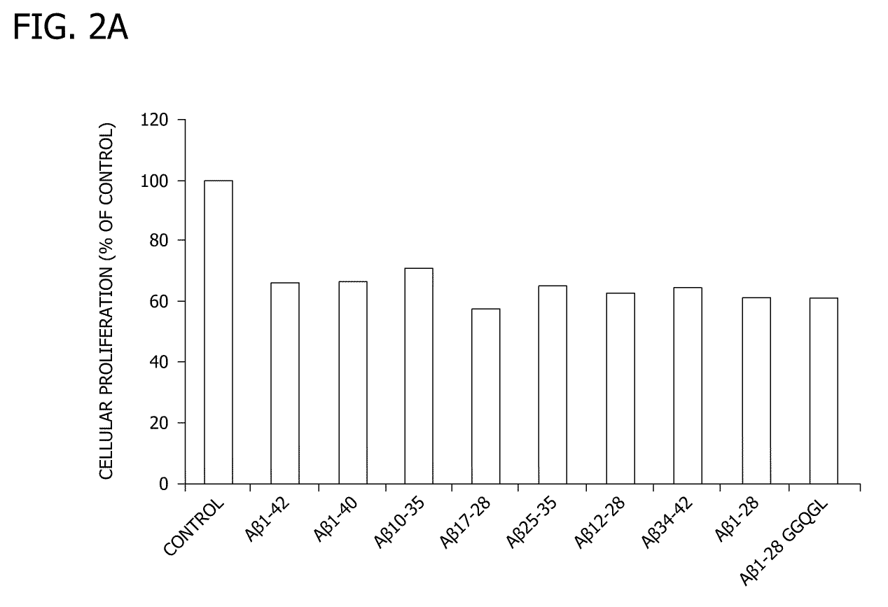

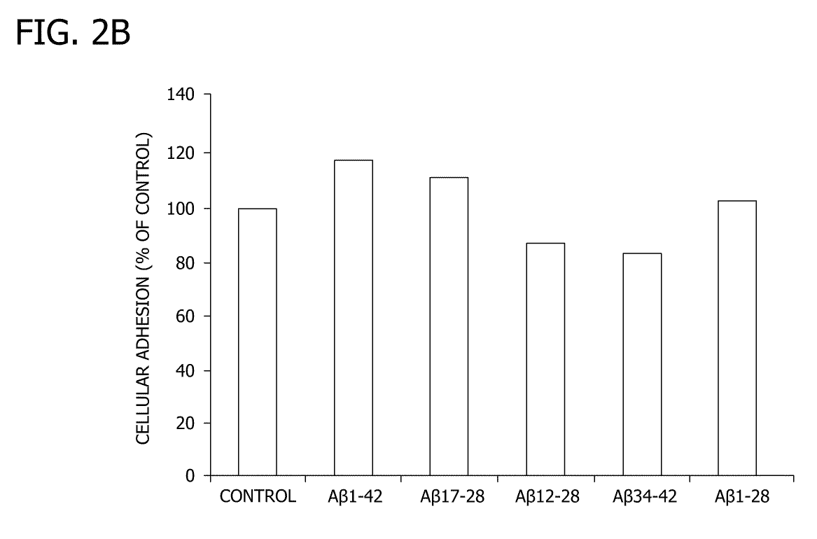

All peptide treatments significantly inhibited cellular proliferation (P<0.005). ANOVA revealed no significant main effects between any of the peptides tested. Post hoc testing revealed no significant differences between the different peptides (P>0.005). Whilst all the fragments tested were able to inhibit cell proliferation of HUVEC, there were no appreciable differences in potency between the different peptides (FIG. 2a). However, both ANOVA and post hoc testing revealed that none of the fragments tested were able to significantly affect cellular adhesion to a basement membrane complex comprising laminin, collagen IV, heparan sulfate proteoglycans and entactin (P>0.005) (FIG. 2b). The differences in anti-angiogenic activity of the Aβ peptide fragments could therefore not be related to effects on cellular proliferation or adhesion.

### Example 10

### Effect of Heparin on Capillary Tube Formation

In order to verify the importance of the putative heparin binding sequence within the Aβ peptide, heparin was added to the samples and the anti-angiogenic activity of Aβ peptide was quantified using the capillary tube formation assay described in Example 3.

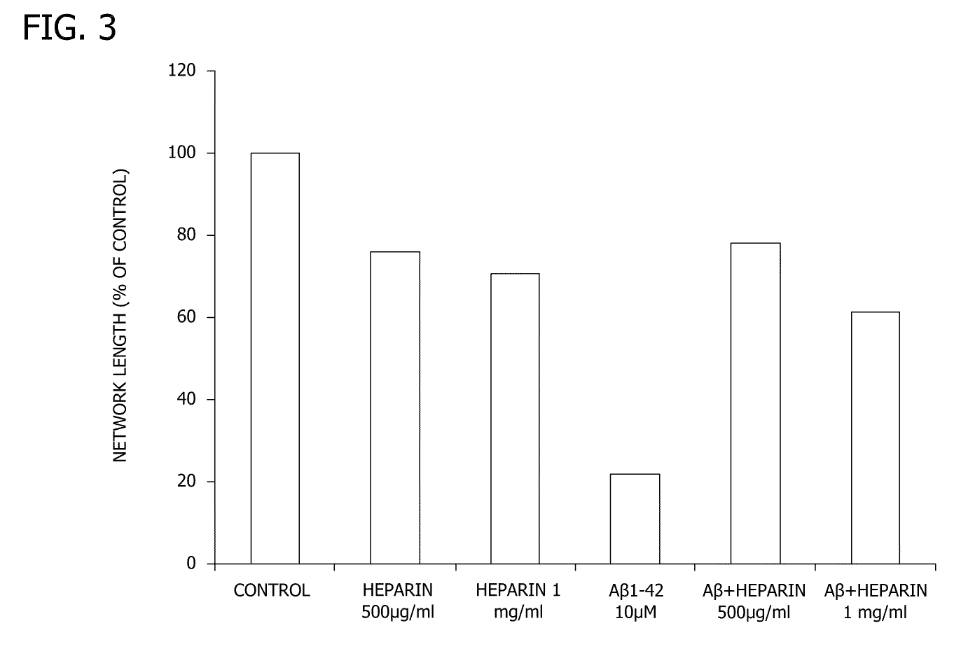

Total length of capillary tubes was quantified for each treatment group (n≧8), and expressed as a percentage of control treatment. Post hoc analysis revealed significant differences between control and all treatment groups (P<0.001), between Aβ and Aβ+heparin 500 μg/ml (P<0.001), Aβ and Aβ+heparin 1 mg/ml (P<0.001). The addition of 500 μg/ml and 1 mg/ml of heparin effectively reversed inhibition of capillary tube formation induced by Aβ₁₋₄₂ (FIG. 3). Addition of heparin alone also caused a slight inhibition of angiogenesis.

### Example 11

### Effect of Proteoglycan Binding Region Mutant Aβ Peptide Fragments on Capillary Tube Formation

Since the addition of heparin reversed the anti-angiogenic activity of Aβ₁₋₄₂, it was hypothesized that the proteoglycan binding region within the peptide may be critical for imparting anti-angiogenic activity. To test this hypothesis, amino acid substitutions, that are known to effectively prevent the binding of Aβ to heparan sulfate proteoglycans (substitution of three amino acids present in the HHQK proteoglycan binding motif for either GGQG, or AAQA) (McLaurin et al. Eur. J. Biochem. 2000, 267, 6353-61; Olofssen, et al. J. Biol. Chem 2005, October 7, advance electronic publication), were made to one of anti-angiogenic peptide fragments (Aβ₁₋₂₈). The effect of the mutant Aβ peptide fragments were then tested in the capillary tube formation assay described in Example 3.

Total length of capillary tubes was quantified for each treatment group (n≧8), and expressed as a percentage of control. ANOVA revealed significant dose dependent main effects of wildtype Aβ₁₋₂₈ (P<0.001), but no main effect of Aβ₁₋₂₈ GGQGL (P=0.566), or Aβ₁₋₂₈ AAQAL (P=0.380). Post hoc analysis revealed significant effects of wildtype Aβ₁₋₂₈ at 1, 5 and 10 μM (P<0.005), but no significant effects of the mutant Aβ₁₋₂₈ peptides at any of the doses tested.

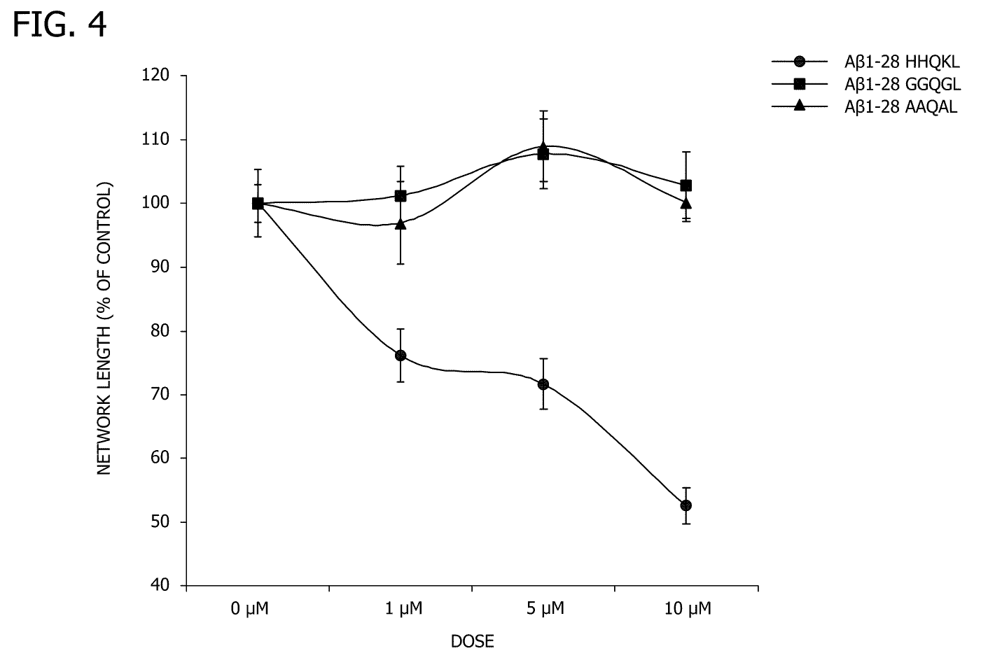

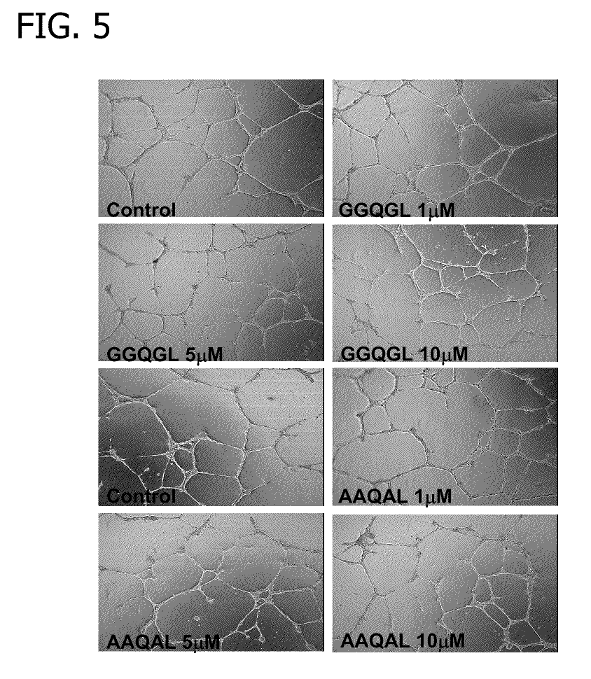

Amino acid substitutions in the proteoglycan binding region of Aβ completely abolished the anti-angiogenic activity of the Aβ₁₋₂₈ peptide (FIG. 4) (see Table 2 for a list of peptide sequences).

TABLE 2

Summary of anti-angiogenic activity of Aβ peptide sequences
at 10 μM

| | | Anti- |
|---|---|---|
| Peptide | Amino Acid Sequence | angiogenic? |
| Aβ₁₋₄₂ | DAEFRHDSGYEVHHQKLVFFAEDVGSNKGAIIGLMVGGVVIA | Y |
| Aβ₁₋₄₀ | DAEFRHDSGYEVHHQKLVFFAEDVGSNKGAIIGLMVGGVV | Y |
| Aβ₁₂₋₂₈ | VHHQKLVFFAEDVGSNK | Y |
| Aβ₁₇₋₂₈ | LVFFAEDVGSNK | N |
| Aβ₁₀₋₃₅ | YEVHHQKLVFFAEDVGSNKGAIIGLM | Y |
| Aβ₂₅₋₃₅ | GSNKGAIIGLM | N |
| Aβ₁₀₋₁₆ | YEVHHQK | N |
| Aβ₃₄₋₄₂ | LMVGGVVIA | N |
| Aβ₁₋₂₈ | DAEFRHDSGYEVHHQKLVFFAEDVGSNK | Y |
| Wildtype | | |
| Aβ₁₋₂₈ | DAEFRHDSGYEVGGQGLVFFAEDVGSNK | N |
| Mutant 1 | | |
| Aβ₁₋₂₈ | DAEFRHDSGYEVAAQALVFFAEDVGSNK | N |
| Mutant 2 | | |
| Fragment | HHHQKLVFF | Y |
| 1 | | |
| Fragment | VHHQKLVII | N |
| 2 | | |
| Fragment | VHHQKLVKK | N |
| 3 | | |

### Example 12

### Effect of LVFF Mutant Aβ Peptide Fragments on Capillary Tube Formation

The role of the VFF amino acid sequence adjacent on the C-terminal side of the HHQK sequence was established by testing peptide fragments consisting of 9 amino acids starting at the HHQK sequence (table 2, fragments 1-3) in the capillary tube formation assay described in Example 3.

Total length of capillary tubes was quantified for each treatment group (n≧6), and expressed as a percentage of control. ANOVA revealed significant main effect for the wildtype (HHHQKLVFF), but not for the mutant peptides. Tumor volumes were measured with an electronic caliper using the formula (length×width×width)/2 where length is the longest axis and width the measurement at right angles to the length (Clarke et al. 2000 Clin Cancer Res. 6, 3621-3628). Post hoc analysis revealed significant effects of wildtype peptide at 1, 5 and 10 μM (P<0.005), but no significant effects of the LVFF mutant peptides at any of the doses tested.

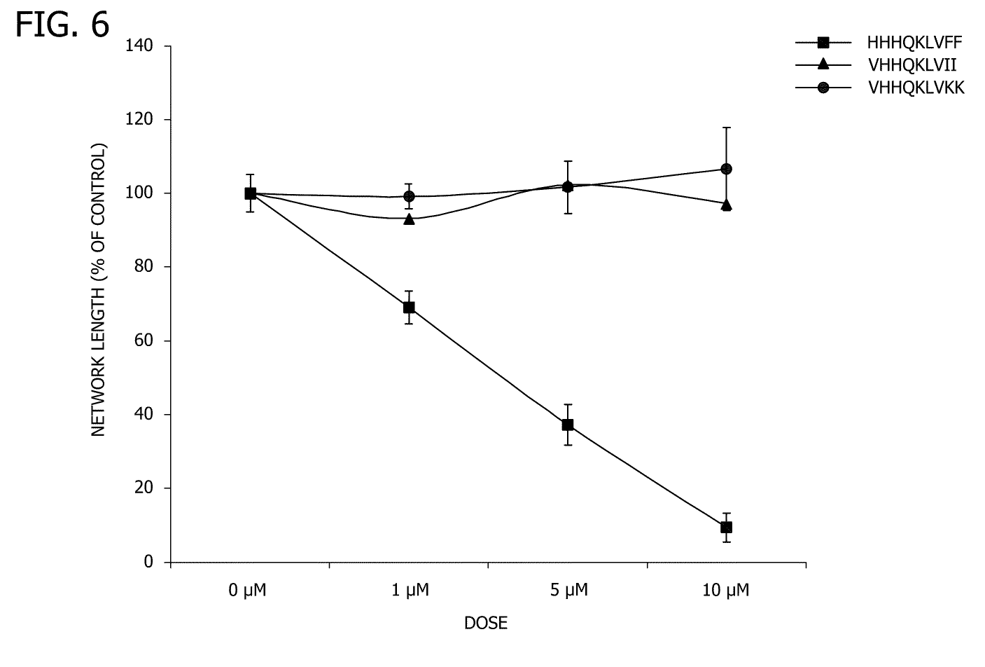

These results support that only a peptide sequence containing both the HHQK and the VFF motif effectively inhibits angiogenesis in capillary tube formation assay (FIG. 6). However, the Aβ₁₀₋₁₆ fragment containing only the YEVHHQK sequence is inactive, showing that this region alone is not sufficient for anti-angiogenic activity.

### Example 13

### Effect of Aβ₁₂₋₂₈ Peptide Fragments in the Rat Corneal Micropocket Assay

In order to determine whether Aβ₁₂₋₂₈ peptide fragment that appeared to be anti-angiogenic in-vitro was also anti-angiogenic in-vivo, Aβ₁₂₋₂₈ was tested in the rat corneal micropocket assay described in Example 6. Corneal micropockets were incubated for 7 days. Quantification of data from the rat corneal micropocket assay in response to 200 ng VEGF, VEGF+0.5 μg Aβ₁₂₋₂₈, VEGF+2.5 μg Aβ₁₂₋₂₈ and VEGF+5.0 μg Aβ₁₂₋₂₈. ANOVA revealed significant main effect of Aβ dose and post hoc analysis revealed a significant effect at the 5 μg dose (P<0.001). Angiogenesis indexes are represented as mean +/−SEM.

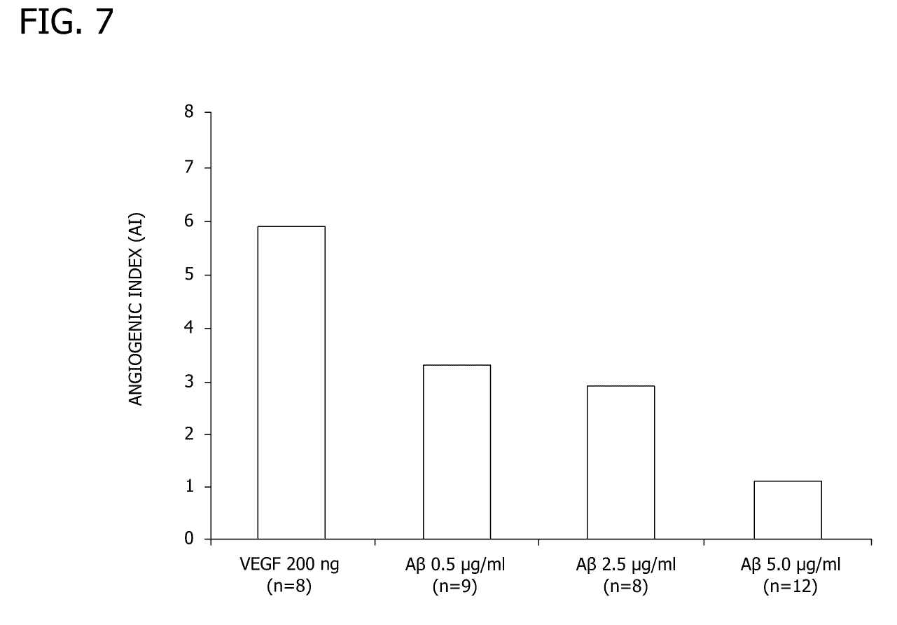

These results support that Aβ₁₂₋₂₈ is able to dose dependently inhibit VEGF-induced angiogenesis in this in-vivo assay (FIG. 7), confirming data from in-vitro experiments.

### Example 14

### Effect of Aβ₁₂₋₂₈, Aβ₁₂₋₂₈ Mutants and Aβ₁₃₋₂₀ in the Rat Corneal Micropocket Assay

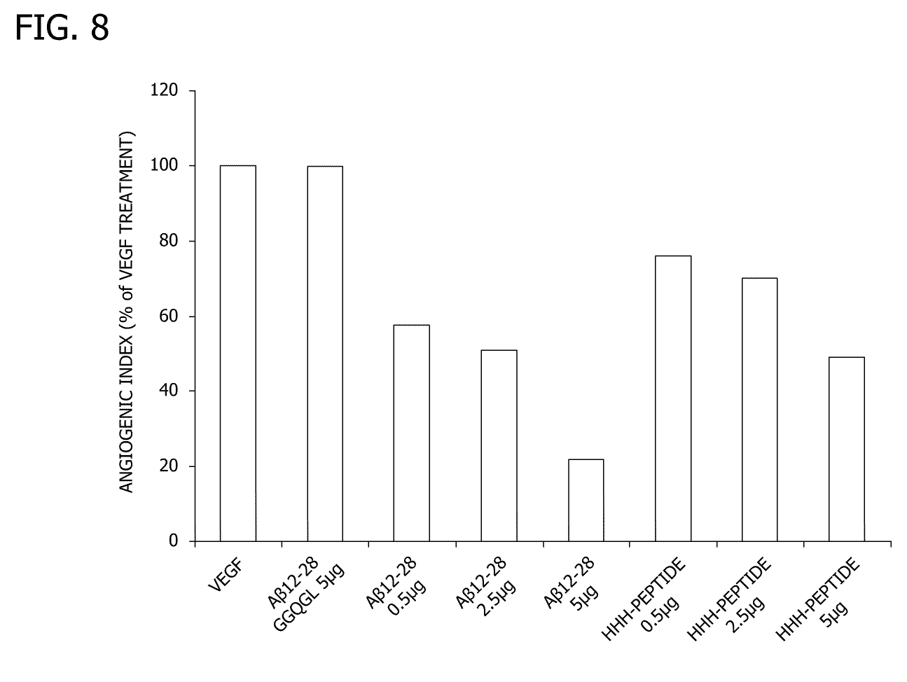

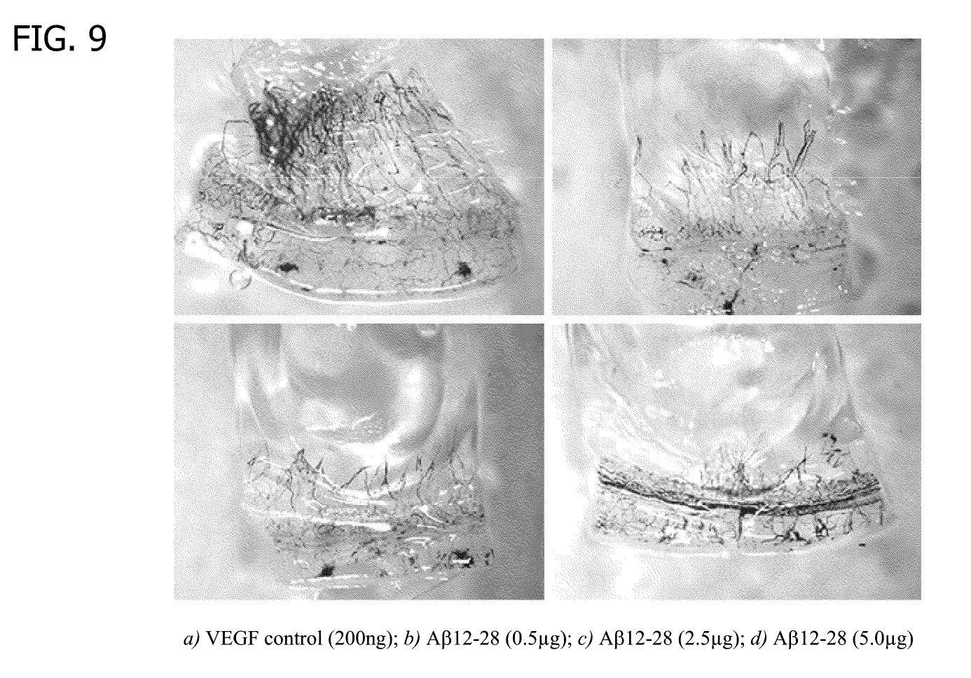

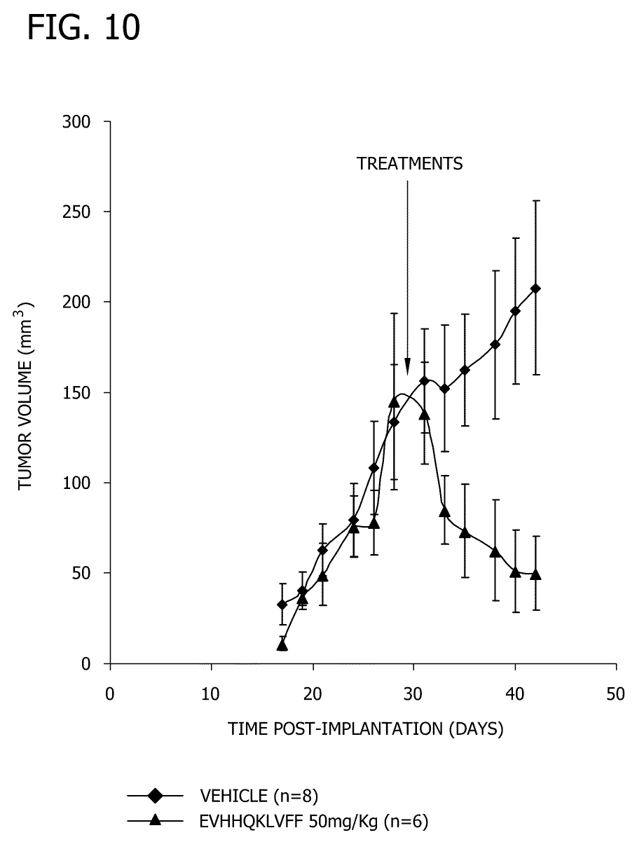

Following the rat corneal micropocket assay method described in Example 6, the effect of various Aβ peptide fragments and mutants was tested. Quanitification of data from the assay in response to response to 200 ng VEGF, 5.0 μg of the Aβ₁₂₋₂₈ GGQGL mutant peptide and 0.5 μg, 2.5 μg and 5.0 μg of Aβ₁₂₋₂₈ and Aβ₁₃₋₂₀ (HHH-peptide or HHQKLVFF). Aβ₁₂₋₂₈ GGQGL mutant is inactive at inhibiting angiogenesis in vivo. The shorter HHH-peptide appears antiangiogenic in vivo (P<0.05 in a dose dependent manner). Results are shown in FIG. 8. 4× magnified photographs of the capillaries are shown in FIG. 9.

### Example 15

### Effect of the Peptide EVHHQKLVFF on the Growth of MCF-7 Human Breast Tumor Xenografts in Nude Mice

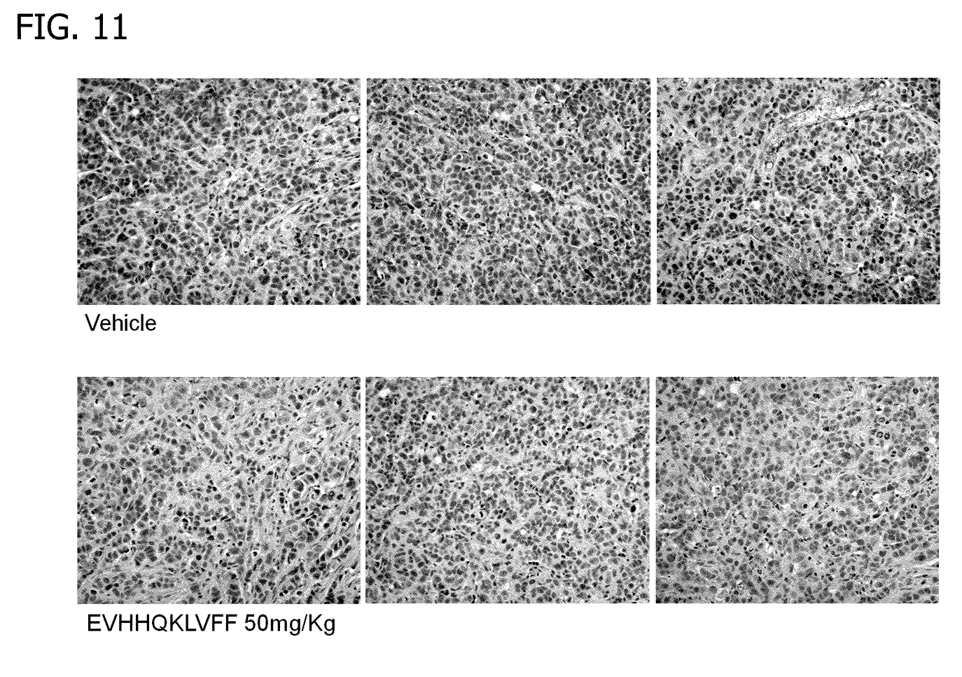

MCF-7 human breast cancer cells were cultured in DMEM medium containing 10% fetal bovine serum and 1× penicillin-streptomycin-fungizone mixture. Female, 8 weeks-old Nu/Nu athymic nude mice (purchased from Harland Teklad, WI) were acclimated in the laboratory 1 week before experimentation. The animals were housed in microisolator cages, four per cage, in a 12-h light/dark cycle. The animals received filtered sterilized water and sterile rodent food ad libitum. To support the growth of the estrogen-dependent MCF-7 tumors, a 1.7 mg 17-β-estradiol 90-day release pellet (Innovative Research of America, Sarasota, Fla.) was implanted subcutaneously a week before the implantation of MCF-7 tumor cells. 3.3 millions of MCF-7 cells were injected subcutaneously into the right and left flank of the nude mice. The tumors were allowed to reach 150 mm³ before the start of the treatment and then animals were randomly divided in two treatment groups. Animals were treated with 50 mg/Kg of body weight of the peptide EVHHQKLVFF or 100 μL of vehicle only (DMSO) once daily by intraperitoneal injection. Mice were injected with MCF-7 human breast tumor xenografts and tumor volume measured. Tumor volumes were measured with an electronic caliper using the formula (length×width×width)/2 where length is the longest axis and width the measurement at right angles to the length (Clarke et al. 2000 Clin Cancer Res. 6, 3621-3628). When the tumors reached a volume of 150 mm³ (32 days) some animals were injected intraperitoneally with the vehicle only (100 microL of DMSO) or with 50 mg/Kg of body weight of the peptide EVHHQKLVFF. Tumor volume was measured to 42 days and mice sacrificed. Results are shown in FIG. 11. As can be seen, tumor volume decreased from approximately 144 mm³ to 50 mm³ between days 28 and 42 in the group treated with the peptide fragment whereas the control group increased from 133 mm3 to 207 mm³.

### Example 16

### PECAM-1 Immunostaining Showing Effect of Peptide EVHHQKLVFF on the Vascularization of Breast Tumors

Mice were injected with MCF-7 human breast tumor xenografts and tumor volume measured as described in Example 15. When the tumors reached a volume of 150 mm³ (32 days) some animals were injected intraperitoneally with the vehicle only (100 microL of DMSO) or with 50 mg/Kg of body weight of the peptide EVHHQKLVFF. Mice were sacrificed at day 42 after tumor implantation and PECAM-1 immunostaining of breast tumor sections was performed. FIG. 12 shows images that a reduction in the vascularization (brown staining) of breast tumors in animals treated with the peptide EVHHQKLVFF compared to animals treated with the vehicle only.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description and shown in the accompanying drawing(s) shall be interpreted as illustrative and not in a limiting sense.

1. An anti-angiogenic Aβ peptide fragment, variant or homolog thereof, wherein the fragment is between 8 and 39 amino acids in length.

2-9. (canceled)

10. The anti-angiogenic Aβ peptide fragment, variant or homolog thereof of claim 1, wherein the variant contains at least one amino acid substitution, the substitution comprising a non-natural amino acid or an amino acid analog.

11. The anti-angiogenic Aβ peptide fragment, variant or homolog thereof of claim 10, wherein the non-natural amino acid is a D amino acid.

12. The anti-angiogenic Aβ peptide fragment, variant or homolog thereof of claim 11, wherein the D amino acid is selected from the group consisting of 3,4-Dehydro-DL-proline; 5-Benzyloxy-DL-tryptophan; D-Alanyl-D-alanine; D-Alanyl-L-leucine; D-Arginine Hydrochloride; D-Asparagine; D-Asparagine, Monohydrate; D-Cystine; D-methionine; D-tryptophan; D-phenylalanine; DL-Alanyl-DL-leucine; DL-Alanyl-DL-leucylglycine; DL-Alanyl-DL-phenylalanine; DL-Arginine Hydrochloride; DL-Cysteine; DL-Cysteine Hydrochloride; DL-Cysteine Hydrochloride Monohydrate; DL-Histidine Hydrochloride, Monohydrate; N-Acetyl-D-leucine; N-Benzoyl-DL-methionine; N-Benzoyl-L-phenylalanine; N-Carbamyl-DL-alanine; N-Chloroacetyl-DL-phenylalanine; N-Chloroacetyl-DL-valine; O-Benzyl-D-serine; O-Benzyl-DL-serine; 3-iodo-L-tyrosine (IY) and p-benzoyl-L-phenylalanine (pBpa).

13. The anti-angiogenic Aβ peptide fragment, variant or homolog thereof of claim 10, wherein the non-natural amino acid has the formula

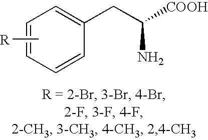

14. The anti-angiogenic Aβ peptide fragment, variant or homolog thereof of claim 10, wherein the non-natural amino acid is a synthetic amino acid.

15. The anti-angiogenic Aβ peptide fragment, variant or homolog thereof of claim 14, wherein the synthetic amino acid has the structure

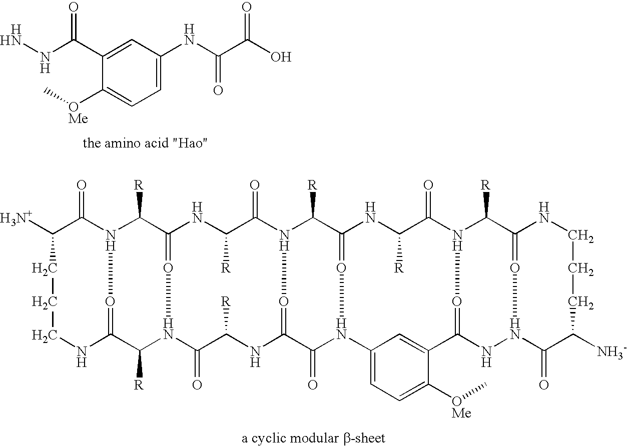

16. The anti-angiogenic Aβ peptide fragment, variant or homolog thereof of claim 14, wherein the synthetic amino acid is selected from the group consisting of L-2-aminohexanoic acid (Ahx), 3-iodo-L-tyrosine, ethylenediaminetetraacetic acid (EDTA)-derivatized tryptophan (Trp), 7-azatryptophan (7AW) and 5-hydroxytryptophan (SHW).

17. The anti-angiogenic Aβ peptide fragment, variant or homolog thereof of claim 10, wherein the non-natural amino acid is a C^{α,α}-disubstituted amino acid.

18. The anti-angiogenic Aβ peptide fragment, variant or homolog thereof of claim 17, wherein the C^{α,α}-disubstituted amino acid is a α-Trifluoromethyl substituted amino acid.

19. The anti-angiogenic Aβ peptide fragment, variant or homolog thereof of claim 10, wherein a statine (3S,4S-4-amino-3-hydroxy-6-methylheptanoic acid) or AHPPA (3S,4S-4-amino-3-hydroxy-5-phenylpentanoic acid) are substituted for any two amino acids of the Aβ peptide fragment, variant or homolog thereof.

20. The anti-angiogenic Aβ peptide fragment, variant or homolog thereof of claim 10, wherein the variant contains from 1 to 10 amino acid substitutions.

21. The anti-angiogenic Aβ peptide fragment, variant or homolog thereof of claim 1, wherein the anti-angiogenic Aβ peptide fragment, variant or homolog thereof is amidated or acetylated at N-terminus, C-terminus or both N- and C-terminus.

22. The anti-angiogenic Aβ peptide fragment, variant or homolog thereof of claim 1, wherein the anti-angiogenic Aβ peptide fragment, variant or homolog thereof contains peptide backbone modification selected from the group consisting of N-methyl, ketomethylene, hydroxyethylene, (E)-ethylene, reduced amide, ether and carba modification.

23. The anti-angiogenic Aβ peptide fragment, variant or homolog thereof of claim 1, wherein anti-angiogenic Aβ peptide fragment, variant or homolog thereof is attached to a ligand selected from the group consisting of a lectin, toxin, viral haemagglutinin, invasin, transferrin, Vitamin B12, folate, riboflavin, biotin, TAT (48-60) peptide, penetratin peptide and oligoarginine peptide.

24. (canceled)

25. The anti-angiogenic Aβ peptide fragment, variant or homolog thereof of claim 1, further comprising a linker, wherein the linker is selected from pyrrolidone, a pyran copolymer, polyhydroxypropylmethacrylamidephenol, polyhydroxy-ethylaspartamidephenol, a polyethyleneoxidepolylysine substituted with palmitoyl residues, polyethylene glycol (PEG), polylactic acid, polyepsilon caprolactone, polyhydroxy butyric acid, a polyorthoester, a polyacetal, a polydihydro-pyran, a polycyanoacrylate, a polyamine, a polyamide, a polyether, a cross-linked copolymer of a hydrogel, and an amphipathic block copolymer of a hydrogel.

26. The anti-angiogenic Aβ peptide fragment, variant or homolog thereof of claim 25, wherein the linker is polyethylene glycol (PEG).

27. The anti-angiogenic Aβ peptide fragment, variant or homolog thereof of claim 1, wherein the anti-angiogenic Aβ peptide fragment, variant or homolog thereof is contained in a liposome.

28. The anti-angiogenic Aβ peptide fragment, variant or homolog thereof of claim 27, wherein the liposome is selected from the group consisting of a small unilamellar vesicle, large unilamellar vesicle and multilamellar vesicle.

29. The anti-angiogenic Aβ peptide fragment, variant or homolog thereof of claim 1, wherein the anti-angiogenic Aβ peptide fragment, variant or homolog thereof is contained in a solid lipid nanoparticle.

30. The anti-angiogenic Aβ peptide fragment, variant or homolog thereof of claim 1, wherein the anti-angiogenic Aβ peptide fragment, variant or homolog thereof is conjugated to a transport vector for receptor-mediated transport or carrier-mediated transport.

31. (canceled)

32. A pharmaceutical composition comprising the anti-angiogenic Aβ peptide fragment, variant or homolog thereof of claim 1 and one or more pharmaceutically acceptable carriers, diluents, or excipients.

33. (canceled)

34. (canceled)

35. A method of treating a disease or disorder mediated by pathological angiogenesis comprising administering to a subject in need thereof an effective amount of an anti-angiogenic Aβ peptide fragment, variant or homolog thereof of claim 1.

36-54. (canceled)

55. A method of treating cancer comprising administering to a subject in need thereof an effective amount of an anti-angiogenic Aβ peptide fragment, variant or homolog thereof of claim 1.

56-66. (canceled)

67. A method for identifying compounds that interfere with Aβ-induced angiogenesis inhibition, comprising (a) contacting a first biological sample capable of undergoing angiogenesis with a test compound, a biologically active amount of an Aβ peptide fragment, variant or homolog thereof, and an angiogenic agent; and (b) determining the extent of angiogenesis that occurs in the first biological sample.

68. (canceled)

69. A method for identifying compounds that interfere with Aβ-induced angiogenesis inhibition, comprising (a) contacting a first biological sample capable of undergoing angiogenesis with a test compound, an anti-angiogenic Aβ peptide fragment, variant or homolog thereof of claim 1, and an angiogenic agent; and (b) determining the extent of angiogenesis that occurs in the first biological sample.

70. The method of claim 69, further comprising (c) separately contacting a second biological sample capable of undergoing angiogenesis with the anti-angiogenic Aβ peptide fragment, variant or homolog thereof and the angiogenic agent; (d) determining the extent of angiogenesis that occurs in the second biological sample; and (e) comparing the extent of angiogenesis that occurs in the first biological sample with that which occurs in the second biological sample.

71. The method of claim 1 wherein the fragment is the amino acid sequence EVHHQKLVFF or an anti-angiogenic variant or homolog thereof.

72. The method of claim 35 wherein the fragment is the amino acid sequence EVHHQKLVFF or an anti-angiogenic variant or homolog thereof.

73. The method of claim 55 wherein the fragment is the amino acid sequence EVHHQKLVFF or an anti-angiogenic variant or homolog thereof.


SEQUENCE LISTING (104 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 42 aa, Homo sapiens [sequence omitted]

SEQ ID NO: 3 - protein, 40 aa, Homo sapiens
DAEFRHDSGYEVHHQKLVFFAEDVGSNKGAIIGLMVGGVV

SEQ ID NO: 4 - protein, 40 aa, Artificial Sequence
  Chemically synthesized; Scrambled A-beta 1-40 peptide (Homo sapiens)
VIGKYHGMSNLVGRSFEVHQGKGAEVDAHGLFDIEAFVDV

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - DNA, 3579 bp, Homo sapiens
agtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagc

SEQ ID NO: 7 - protein, 42 aa, Artificial Sequence
  Chemically synthesized; Synthetic 21G-A-beta 1-42 (Flemish) mutant peptide (Homo sapiens)
DAEFRHDSGYEVHHQKLVFFGEDVGSNKGAIIGLMVGGVVIA

SEQ ID NO: 8 - protein, 42 aa, Artificial Sequence
  Chemically synthesized; Synthetic 22Q-A-beta 1-42 (Dutch) mutant peptide (Homo sapiens)
DAEFRHDSGYEVHHQKLVFFAQDVGSNKGAIIGLMVGGVVIA

SEQ ID NO: 9 - protein, 42 aa, Artificial Sequence
  Chemically synthesized; Synthetic 22K-A-beta 1-42 (Italian) mutant peptide (Homo sapiens)
DAEFRHDSGYEVHHQKLVFFAKDVGSNKGAIIGLMVGGVVIA

SEQ ID NO: 10 - protein, 42 aa, Artificial Sequence
  Chemically synthesized; Synthetic 22G-A-beta 1-42 (Arctic) mutant peptide (Homo sapiens)
DAEFRHDSGYEVHHQKLVFFAGDVGSNKGAIIGLMVGGVVIA

SEQ ID NO: 11 - protein, 42 aa, Artificial Sequence; identical to SEQ ID NO: 10
  Chemically synthesized; Synthetic 23N-A-beta 1-42 (Iowa) mutant peptide (Homo sapiens)

SEQ ID NO: 12 - protein, 10 aa, Artificial Sequence
  Chemically synthesized; Amyloid-Beta (Abeta) peptide fragment (Homo sapiens)
EVHHQKLVFF

SEQ ID NO: 13 - protein, 4 aa, Artificial Sequence
  Chemically synthesized; A proteoglycan binding region of Abeta (Homo sapiens)
HHQK

SEQ ID NO: 14 - protein, 4 aa, Artificial Sequence
  Chemically synthesized; The region immediately following the HHQK domain of Abeta (Homo sapiens)
LVFF

SEQ ID NO: 15 - protein, 8 aa, Homo sapiens
HHQKLVFF

SEQ ID NO: 16 - protein, 8 aa, Mus musculus
RHQKLVFF

SEQ ID NO: 17 - protein, 8 aa, Artificial Sequence
  Chemically synthesized; Amyloid-Beta (Abeta) peptide fragment variant (Homo sapiens)
HHNKLVFF

SEQ ID NO: 18 - protein, 8 aa, Artificial Sequence
  Chemically synthesized; Amyloid-Beta (Abeta) peptide fragment variant (Homo sapiens)
RHNKLVFF

SEQ ID NO: 19 - protein, 8 aa, Artificial Sequence
  Chemically synthesized; Amyloid-Beta (Abeta) peptide fragment variant (Homo sapiens)
HHQRLVFF

SEQ ID NO: 20 - protein, 8 aa, Artificial Sequence
  Chemically synthesized; Amyloid-Beta (Abeta) peptide fragment variant (Homo sapiens)
RHQRLVFF

SEQ ID NO: 21 - protein, 8 aa, Artificial Sequence
  Chemically synthesized; Amyloid-Beta (Abeta) peptide fragment variant (Homo sapiens)
HHNRLVFF

SEQ ID NO: 22 - protein, 8 aa, Artificial Sequence
  Chemically synthesized; Amyloid-Beta (Abeta) peptide fragment variant (Homo sapiens)
RHNRLVFF

SEQ ID NO: 23 - protein, 8 aa, Artificial Sequence
  Chemically synthesized; Amyloid-Beta (Abeta) peptide fragment variant (Homo sapiens)
HHQKIVFF

SEQ ID NO: 24 - protein, 8 aa, Artificial Sequence
  Chemically synthesized; Amyloid-Beta (Abeta) peptide fragment variant (Homo sapiens)
RHQKIVFF

SEQ ID NO: 25 - protein, 8 aa, Artificial Sequence
  Chemically synthesized; Amyloid-Beta (Abeta) peptide fragment variant (Homo sapiens)
HHNKIVFF

SEQ ID NO: 26 - protein, 8 aa, Artificial Sequence
  Chemically synthesized; Amyloid-Beta (Abeta) peptide fragment variant (Homo sapiens)
RHNKIVFF

SEQ ID NO: 27 - protein, 8 aa, Artificial Sequence
  Chemically synthesized; Amyloid-Beta (Abeta) peptide fragment variant (Homo sapiens)
HHQRIVFF

SEQ ID NO: 28 - protein, 8 aa, Geobacillus stearothermophilus
RHQRIVFF

SEQ ID NO: 29 - protein, 8 aa, Artificial Sequence
  Chemically synthesized; Amyloid-Beta (Abeta) peptide fragment variant (Homo sapiens)
HHNRIVFF

SEQ ID NO: 30 - protein, 8 aa, Artificial Sequence
  Chemically synthesized; Amyloid-Beta (Abeta) peptide fragment variant (Homo sapiens)
RHNRIVFF

SEQ ID NO: 31 - protein, 8 aa, Artificial Sequence
  Chemically synthesized; Amyloid-Beta (Abeta) peptide fragment variant (Homo sapiens)
HHQKVVFF

SEQ ID NO: 32 - protein, 8 aa, Artificial Sequence
  Chemically synthesized; Amyloid-Beta (Abeta) peptide fragment variant (Homo sapiens)
RHQKVVFF

SEQ ID NO: 33 - protein, 8 aa, Artificial Sequence
  Chemically synthesized; Amyloid-Beta (Abeta) peptide fragment variant (Homo sapiens)
HHNKVVFF

SEQ ID NO: 34 - protein, 8 aa, Artificial Sequence
  Chemically synthesized; Amyloid-Beta (Abeta) peptide fragment variant (Homo sapiens)
RHNKVVFF

SEQ ID NO: 35 - protein, 8 aa, Yarrowia lipolytica
HHQRVVFF

SEQ ID NO: 36 - protein, 8 aa, Artificial Sequence
  Chemically synthesized; Amyloid-Beta (Abeta) peptide fragment variant (Homo sapiens)
RHQRVVFF

SEQ ID NO: 37 - protein, 8 aa, Artificial Sequence
  Chemically synthesized; Amyloid-Beta (Abeta) peptide fragment variant (Homo sapiens)
HHNRVVFF

SEQ ID NO: 38 - protein, 8 aa, Artificial Sequence
  Chemically synthesized; Amyloid-Beta (Abeta) peptide fragment variant (Homo sapiens)
RHNRVVFF

SEQ ID NO: 39 - protein, 8 aa, Artificial Sequence
  Chemically synthesized; Amyloid-Beta (Abeta) peptide fragment variant (Homo sapiens)
HHQKLIFF

SEQ ID NO: 40 - protein, 8 aa, Artificial Sequence
  Chemically synthesized; Amyloid-Beta (Abeta) peptide fragment variant (Homo sapiens)
RHQKLIFF

SEQ ID NO: 41 - protein, 8 aa, Artificial Sequence
  Chemically synthesized; Amyloid-Beta (Abeta) peptide fragment variant (Homo sapiens)
HHNKLIFF

SEQ ID NO: 42 - protein, 8 aa, Artificial Sequence
  Chemically synthesized; Amyloid-Beta (Abeta) peptide fragment variant (Homo sapiens)
RHNKLIFF

SEQ ID NO: 43 - protein, 8 aa, Artificial Sequence
  Chemically synthesized; Amyloid-Beta (Abeta) peptide fragment variant (Homo sapiens)
HHQRLIFF

SEQ ID NO: 44 - protein, 8 aa, Artificial Sequence
  Chemically synthesized; Amyloid-Beta (Abeta) peptide fragment variant (Homo sapiens)
RHQRLIFF

SEQ ID NO: 45 - protein, 8 aa, Artificial Sequence
  Chemically synthesized; Amyloid-Beta (Abeta) peptide fragment variant (Homo sapiens)
HHNRLIFF

SEQ ID NO: 46 - protein, 8 aa, Artificial Sequence
  Chemically synthesized; Amyloid-Beta (Abeta) peptide fragment variant (Homo sapiens)
RHNRLIFF

SEQ ID NO: 47 - protein, 8 aa, Artificial Sequence
  Chemically synthesized; Amyloid-Beta (Abeta) peptide fragment variant (Homo sapiens)
HHQKIIFF

SEQ ID NO: 48 - protein, 8 aa, Artificial Sequence
  Chemically synthesized; Amyloid-Beta (Abeta) peptide fragment variant (Homo sapiens)
RHQKIIFF

SEQ ID NO: 49 - protein, 8 aa, Artificial Sequence
  Chemically synthesized; Amyloid-Beta (Abeta) peptide fragment variant (Homo sapiens)
HHNKIIFF

SEQ ID NO: 50 - protein, 8 aa, Artificial Sequence
  Chemically synthesized; Amyloid-Beta (Abeta) peptide fragment variant (Homo sapiens)
RHNKIIFF

SEQ ID NO: 51 - protein, 8 aa, Artificial Sequence
  Chemically synthesized; Amyloid-Beta (Abeta) peptide fragment variant (Homo sapiens)
HHQRIIFF

SEQ ID NO: 52 - protein, 8 aa, Artificial Sequence
  Chemically synthesized; Amyloid-Beta (Abeta) peptide fragment variant (Homo sapiens)
RHQRIIFF

SEQ ID NO: 53 - protein, 8 aa, Artificial Sequence
  Chemically synthesized; Amyloid-Beta (Abeta) peptide fragment variant (Homo sapiens)
HHNRIIFF

SEQ ID NO: 54 - protein, 8 aa, Artificial Sequence
  Chemically synthesized; Amyloid-Beta (Abeta) peptide fragment variant (Homo sapiens)
RHNRIIFF

SEQ ID NO: 55 - protein, 8 aa, Artificial Sequence
  Chemically synthesized; Amyloid-Beta (Abeta) peptide fragment variant (Homo sapiens)
HHQKVIFF

SEQ ID NO: 56 - protein, 8 aa, Artificial Sequence
  Chemically synthesized; Amyloid-Beta (Abeta) peptide fragment variant (Homo sapiens)
RHQKVIFF

SEQ ID NO: 57 - protein, 8 aa, Artificial Sequence
  Chemically synthesized; Amyloid-Beta (Abeta) peptide fragment variant (Homo sapiens)
HHNKVIFF

SEQ ID NO: 58 - protein, 8 aa, Artificial Sequence
  Chemically synthesized; Amyloid-Beta (Abeta) peptide fragment variant (Homo sapiens)
RHNKVIFF

SEQ ID NO: 59 - protein, 8 aa, Artificial Sequence
  Chemically synthesized; Amyloid-Beta (Abeta) peptide fragment variant (Homo sapiens)
HHQRVIFF

SEQ ID NO: 60 - protein, 8 aa, Artificial Sequence
  Chemically synthesized; Amyloid-Beta (Abeta) peptide fragment variant (Homo sapiens)
RHQRVIFF

SEQ ID NO: 61 - protein, 8 aa, Artificial Sequence
  Chemically synthesized; Amyloid-Beta (Abeta) peptide fragment variant (Homo sapiens)
HHQKLLFF

SEQ ID NO: 62 - protein, 8 aa, Artificial Sequence
  Chemically synthesized; Amyloid-Beta (Abeta) peptide fragment variant (Homo sapiens)
RHQKLLFF

SEQ ID NO: 63 - protein, 8 aa, Artificial Sequence
  Chemically synthesized; Amyloid-Beta (Abeta) peptide fragment variant (Homo sapiens)
HHNKLLFF

SEQ ID NO: 64 - protein, 8 aa, Artificial Sequence
  Chemically synthesized; Amyloid-Beta (Abeta) peptide fragment variant (Homo sapiens)
RHNKLLFF

SEQ ID NO: 65 - protein, 8 aa, Artificial Sequence
  Chemically synthesized; Amyloid-Beta (Abeta) peptide fragment variant (Homo sapiens)
HHQRLLFF

SEQ ID NO: 66 - protein, 8 aa, Artificial Sequence
  Chemically synthesized; Amyloid-Beta (Abeta) peptide fragment variant (Homo sapiens)
RHQRLLFF

SEQ ID NO: 67 - protein, 8 aa, Artificial Sequence
  Chemically synthesized; Amyloid-Beta (Abeta) peptide fragment variant (Homo sapiens)
HHNRLLFF

SEQ ID NO: 68 - protein, 8 aa, Artificial Sequence
  Chemically synthesized; Amyloid-Beta (Abeta) peptide fragment variant (Homo sapiens)
RHNRLLFF

SEQ ID NO: 69 - protein, 8 aa, Artificial Sequence
  Chemically synthesized; Amyloid-Beta (Abeta) peptide fragment variant (Homo sapiens)
HHQKILFF

SEQ ID NO: 70 - protein, 8 aa, Artificial Sequence
  Chemically synthesized; Amyloid-Beta (Abeta) peptide fragment variant (Homo sapiens)
RHQKILFF

SEQ ID NO: 71 - protein, 8 aa, Artificial Sequence
  Chemically synthesized; Amyloid-Beta (Abeta) peptide fragment variant (Homo sapiens)
HHNKILFF

SEQ ID NO: 72 - protein, 8 aa, Artificial Sequence
  Chemically synthesized; Amyloid-Beta (Abeta) peptide fragment variant (Homo sapiens)
RHNKILFF

SEQ ID NO: 73 - protein, 8 aa, Artificial Sequence
  Chemically synthesized; Amyloid-Beta (Abeta) peptide fragment variant (Homo sapiens)
HHQRILFF

SEQ ID NO: 74 - protein, 8 aa, Artificial Sequence
  Chemically synthesized; Amyloid-Beta (Abeta) peptide fragment variant (Homo sapiens)
RHQRILFF

SEQ ID NO: 75 - protein, 8 aa, Artificial Sequence
  Chemically synthesized; Amyloid-Beta (Abeta) peptide fragment variant (Homo sapiens)
HHNRILFF

SEQ ID NO: 76 - protein, 8 aa, Artificial Sequence
  Chemically synthesized; Amyloid-Beta (Abeta) peptide fragment variant (Homo sapiens)
RHNRILFF

SEQ ID NO: 77 - protein, 8 aa, Artificial Sequence
  Chemically synthesized; Amyloid-Beta (Abeta) peptide fragment variant (Homo sapiens)
HHQKVLFF

SEQ ID NO: 78 - protein, 8 aa, Artificial Sequence
  Chemically synthesized; Amyloid-Beta (Abeta) peptide fragment variant (Homo sapiens)
RHQKVLFF

SEQ ID NO: 79 - protein, 8 aa, Artificial Sequence
  Chemically synthesized; Amyloid-Beta (Abeta) peptide fragment variant (Homo sapiens)
HHNKVLFF

SEQ ID NO: 80 - protein, 8 aa, Artificial Sequence
  Chemically synthesized; Amyloid-Beta (Abeta) peptide fragment variant (Homo sapiens)
RHNKVLFF

SEQ ID NO: 81 - protein, 8 aa, Artificial Sequence
  Chemically synthesized; Amyloid-Beta (Abeta) peptide fragment variant (Homo sapiens)
HHQRVLFF

SEQ ID NO: 82 - protein, 8 aa, Artificial Sequence
  Chemically synthesized; Amyloid-Beta (Abeta) peptide fragment variant (Homo sapiens)
RHQRVLFF

SEQ ID NO: 83 - protein, 8 aa, Artificial Sequence
  Chemically synthesized; Amyloid-Beta (Abeta) peptide fragment variant (Homo sapiens)
HHNRVIFF

SEQ ID NO: 84 - protein, 8 aa, Artificial Sequence
  Chemically synthesized; Amyloid-Beta (Abeta) peptide fragment variant (Homo sapiens)
RHNRVIFF

SEQ ID NO: 85 - protein, 42 aa, Homo sapiens [sequence omitted]

SEQ ID NO: 86 - protein, 40 aa, Homo sapiens; identical to SEQ ID NO: 3

SEQ ID NO: 87 - protein, 17 aa, Homo sapiens
VHHQKLVFFAEDVGSNK

SEQ ID NO: 88 - protein, 12 aa, Homo sapiens
LVFFAEDVGSNK

SEQ ID NO: 89 - protein, 26 aa, Homo sapiens
YEVHHQKLVFFAEDVGSNKGAIIGLM

SEQ ID NO: 90 - protein, 11 aa, Homo sapiens
GSNKGAIIGLM

SEQ ID NO: 91 - protein, 7 aa, Homo sapiens
YEVHHQK

SEQ ID NO: 92 - protein, 9 aa, Homo sapiens
LMVGGVVIA

SEQ ID NO: 93 - protein, 28 aa, Homo sapiens
DAEFRHDSGYEVHHQKLVFFAEDVGSNK

SEQ ID NO: 94 - protein, 28 aa, Artificial Sequence
  Chemically synthesized; Abeta1-28  Mutant (Homo sapiens)
DAEFRHDSGYEVGGQGLVFFAEDVGSNK

SEQ ID NO: 95 - protein, 28 aa, Artificial Sequence
  Chemically synthesized; Abeta1-28 mutant (Homo sapiens)
DAEFRHDSGYEVAAQALVFFAEDVGSNK

SEQ ID NO: 96 - protein, 9 aa, Artificial Sequence
  Chemically synthesized; Peptide fragment 1, as described in Table 2 of Example 11 (Homo sapiens)
HHHQKLVFF

SEQ ID NO: 97 - protein, 9 aa, Artificial Sequence
  Chemically synthesized; Peptide fragment 2, as described in Table 2 of Example 11 (Homo sapiens)
VHHQKLVII

SEQ ID NO: 98 - protein, 9 aa, Artificial Sequence
  Chemically synthesized; Peptide fragment 3, as described in Table 2 of Example 11 (Homo sapiens)
VHHQKLVKK

SEQ ID NO: 99 - protein, 7 aa, Artificial Sequence
  Chemically synthesized; Abeta10-16 (Homo sapiens)
YEVHHQK

SEQ ID NO: 100 - protein, 4 aa, Artificial Sequence
  Chemically synthesized; neutral amino acid substitutions for HHQK (Homo sapiens)
AAQA

SEQ ID NO: 101 - protein, 4 aa, Artificial
  Chemically synthesized; Neutral amino acid substitutions for HHQK (Homo sapiens)
GGQG

SEQ ID NO: 102 - protein, 5 aa, Artificial Sequence
  Chemically synthesized; neutral amino acid substitutions for HHQKL (Homo sapiens)
GGQGL

SEQ ID NO: 103 - protein, 5 aa, Artificial Sequence
  Chemically synthesized; neutral amino acid substitutions for HHQKL (Homo sapiens)
AAQAL

SEQ ID NO: 104 - protein, 8 aa, Artificial Sequence
  Abeta peptide fragment variant (Homo sapiens)
XHXXXXFF